SEQUENCE LISTING (20 sequences)

SEQ ID NO: 1 - DNA, 25 bp, Porphyromonas gingivalis
  source 1..25: mol_type = genomic DNA
ggaagagaagaccgtagcacaagga

SEQ ID NO: 2 - DNA, 25 bp, Porphyromonas gingivalis
  source 1..25: mol_type = genomic DNA
gagtaggcgaaacgtccatcaggtc

SEQ ID NO: 3 - DNA, 22 bp, Porphyromonas gingivalis
  source 1..22: mol_type = genomic DNA
ggattgaccaccggcgaagaca

SEQ ID NO: 4 - DNA, 24 bp, Porphyromonas gingivalis
  source 1..24: mol_type = genomic DNA
cggacacgacggttactcaaatgg

SEQ ID NO: 5 - DNA, 20 bp, Porphyromonas gingivalis
  source 1..20: mol_type = genomic DNA
acccattggcagaagttacg

SEQ ID NO: 6 - DNA, 24 bp, Porphyromonas gingivalis
  source 1..24: mol_type = genomic DNA
tcaataggacacaagcgaccatag

SEQ ID NO: 7 - DNA, 24 bp, Porphyromonas gingivalis
  source 1..24: mol_type = genomic DNA
agcagagttccgtcgtcgtattca

SEQ ID NO: 8 - DNA, 20 bp, Porphyromonas gingivalis
  source 1..20: mol_type = genomic DNA
gcgccctgccatcttgtctc

SEQ ID NO: 9 - DNA, 20 bp, Porphyromonas gingivalis
  source 1..20: mol_type = genomic DNA
aaccggagcaaaactgagaa

SEQ ID NO: 10 - DNA, 20 bp, Porphyromonas gingivalis
  source 1..20: mol_type = genomic DNA
ccgtccgccactaacttcta

SEQ ID NO: 11 - DNA, 23 bp, Porphyromonas gingivalis
  source 1..23: mol_type = genomic DNA
cgggcgtgcatcttgtcgtctac

SEQ ID NO: 12 - DNA, 22 bp, Porphyromonas gingivalis
  source 1..22: mol_type = genomic DNA
cttaaccggccgcctctttgaa

SEQ ID NO: 13 - DNA, 22 bp, Porphyromonas gingivalis
  source 1..22: mol_type = genomic DNA
acctaagggagaaacagaacca

SEQ ID NO: 14 - DNA, 22 bp, Porphyromonas gingivalis
  source 1..22: mol_type = genomic DNA
cctgcctttaattcatctccat

SEQ ID NO: 15 - DNA, 19 bp, Porphyromonas gingivalis
  source 1..19: mol_type = genomic DNA
ttggctcaattggctggaa

SEQ ID NO: 16 - DNA, 23 bp, Porphyromonas gingivalis
  source 1..23: mol_type = genomic DNA
gtaatcgtcgtgttcgttctgtg

SEQ ID NO: 17 - DNA, 22 bp, Porphyromonas gingivalis
  source 1..22: mol_type = genomic DNA
ctgggcaaagcgtctggaaaac

SEQ ID NO: 18 - DNA, 24 bp, Porphyromonas gingivalis
  source 1..24: mol_type = genomic DNA
gaaatcacgaatcggggaaatctc

SEQ ID NO: 19 - DNA, 20 bp, synthetic construct
  source 1..20: mol_type = other DNA
gtgstgcayggytgtcgtca

SEQ ID NO: 20 - DNA, 20 bp, synthetic construct
  source 1..20: mol_type = other DNA
acgtcrtccmcaccttcctc

(19) United States
(12) Patent Application Publication
(10) Pub. No.: US 2025/0270658 A1
(43) Pub. Date: Aug. 28, 2025

(54) COMPOSITION FOR DIAGNOSING PERIODONTAL DISEASES BY USING BACTERIAL CLUSTERS IN GINGIVAL CREVICULAR FLUID, AND USE THEREOF

(71) Applicant: AJOU UNIVERSITY INDUSTRY-ACADEMIC COOPERATION FOUNDATION, Suwon-si, Gyeonggi-do (KR)

(72) Inventors: Suk JI, Seoul (KR); Joong Ki KOOK, Gwangju (KR); Soon Nang PARK, Gwangju (KR); Yun Kyong LIM, Gwangju (KR); Jae-Suk JUNG, Suwon-si, Gyeonggi-do (KR); Geum Hee CHOI, Gunpo-si, Gyeonggi-do (KR)

(73) Assignee: AJOU UNIVERSITY INDUSTRY-ACADEMIC COOPERATION FOUNDATION, Suwon-si, Gyeonggi-do (KR)

(21) Appl. No.: 18/700,307

(22) PCT Filed: Oct. 18, 2022
(86) PCT No.: PCT/KR2022/015881

§ 371 Date: Apr. 11, 2024

(30) Foreign Application Priority Data
Oct. 19, 2021 (KR) 10-2021-0138898

(51) Int. Cl. C12Q 1/689

(52) U.S. Cl. CPC C12Q 1/689; C12Q 2600/112; C12Q 2600/158; C12Q 2600/16

(57) ABSTRACT

The present invention relates to a composition for diagnosing periodontal diseases by using bacterial clusters in gingival crevicular fluid, and a use thereof, and the objective of the present invention is to distinguish the severity of periodontal diseases by using bacterial clusters distributed in the gingival crevicular fluid and relative ratios thereof, wherein differences in characteristics of bacterial clusters according to gingivitis, moderate periodontitis and severe periodontitis are exhibited, and thus a standard for distinguishing the severity of periodontal diseases by using same is proposed, and in addition, the present invention is advantageous in that, with respect to sample collection, gingival crevicular fluid is safe, easily and quickly accessible, and allows a non-invasive method to be used so that patient inconvenience can be minimized, and the development of a diagnostic kit using the gingival crevicular fluid can be helpful in identifying the degree of progression of periodontal diseases.

### CROSS REFERENCE TO PRIOR APPLICATIONS

This application is a National Stage Application of PCT International Patent Application No. PCT/KR2022/015881 filed on Oct. 18, 2022, under 35 U.S.C. § 371, which claims priority to Korean Patent Application No. 10-2021-0138898 filed Oct. 19, 2021, respectively, which are all hereby incorporated by reference in their entirety.

### SEQUENCE LISTING

This application contains a Sequence Listing submitted via EFS-Web and hereby incorporated by reference in its entirety. The Sequence Listing is named 2280-515_SEQCRF.xml, created on Apr. 10, 2024, and 17,946 bytes in size.

### TECHNICAL FIELD

The present disclosure relates to a composition for diagnosing a periodontal disease using bacterial colonies in gingival crevicular fluid and a use thereof.

### BACKGROUND ART

A periodontal disease is a chronic disease in which inflammation progresses with an unawareness of the illness by patients to cause severe destruction in tissues around the tooth, and in most cases, it is an outcome of bacterial infiltration and an immune response in a host against bacteria and also the main cause of tooth loss.

Periodontitis is an inflammation occurring in the supportive tissue of teeth and is accompanied by gradual destruction of connective tissues as well as the loss of alveolar bone and periodontal ligaments. Depending on the rate of periodontal destruction, disease activity and host resistance, different types of bacteria are involved. Systemic diseases such as diabetes may aggravate periodontitis while host factors such as medication, smoking, pregnancy, obesity, and excessive stress are also risk factors. In addition, in the case of rapidly progressing periodontitis, it is known that genetic factors play a crucial role.

A method of measuring a probing pocket depth by inserting a probe into the sulcus, one of diagnostic methods currently used to diagnose periodontitis, is the most basic diagnostic method for periodontitis diagnosis, as a method of checking how much alveolar bone has been lost and determining a degree of inflammation in the gingiva. However, the method of measuring the probing pocket depth may cause errors depending on the tooth shape and a degree of gingival inflammation.

A method of radiographically determining the bone loss, along with probes for the probing pocket, is the most basic method of diagnosing periodontitis. However, it has the limitation in that it may show the alveolar bone loss in the mesiodistal surface of the tooth through a two-dimensional image without showing the alveolar bone loss in the buccolingual surface of the tooth where the image overlaps with the tooth. In addition, a method of identifying the probing pocket depth and bone loss on radiographs only shows the outcome of alveolar bone loss (loss of attachment) due to the progression of periodontitis before the time of diagnosis, without showing the active state of the current disease.

On the other hand, currently the most useful method that shows whether the gingiva has inflammation at the time of diagnosis is a method of inserting a probe into the sulcus between the tooth and the gingiva to check for bleeding. However, this method has the limitation in that there is a high probability of a false positive (despite the bleeding on probing, the actual gingiva may not have inflammation).

Such the conventional methods are cumbersome, time-consuming, and costly as the measurement is conducted by a specialist in the doctor's office, and some methods involve pain in the patient. In addition, measurement of probing pocket and clinical attachment level and observation of alveolar bone level on radiographs show the clinical outcome of periodontal diseases without providing information on whether the gum condition of individual teeth is currently causing attachment loss.

The periodontal disease, the main cause of tooth loss, is primarily caused by bacterial infection. Therefore, it is important to reduce the number of bacteria in the oral cavity and actively treat it through early diagnosis. Thereby, there is a need to develop a method that may easily diagnose, for a periodontal disease with late detectable symptoms, whether the current condition of the gum on individual teeth is healthy without inflammation or has periodontitis.

In other words, if severity of gum disease in individual teeth is determined by the concentration of bacteria and patients are able to determine the time of treatment early after checking the presence of a gum disease on their own, the time and cost for the treatment may be dramatically reduced.

### DISCLOSURE OF THE INVENTION

### Technical Goals

An object of the present disclosure is to provide a composition for diagnosing a periodontal disease.

Another object of the present disclosure is to provide a kit for diagnosing a periodontal disease.

Still another object of the present disclosure is to provide a method of providing information necessary for diagnosis of a periodontal disease in gums on individual teeth.

### Technical Solutions

To achieve the above objects, the present disclosure provides a method of providing information necessary for diagnosis of a periodontal disease, including performing quantitative analysis by real-time polymerase chain reaction (real-time PCR) with one or more bacteria selected from the group consisting of Porphyromonas gingivalis, Tannerella forsythia, Prevotella intermedia, Porphyromonas endodontalis, Filifactor alocis, Treponema denticola, and Rothia dentocariosa in a sample isolated from an individual; and comparing a bacterial % or a bacterial count obtained through the quantitative analysis by real-time PCR with that selected from the group consisting of normal individuals and patients with gingivitis, moderate periodontitis, and severe periodontitis.

In addition, the present disclosure provides a method of providing information necessary for diagnosis of a periodontal disease, including performing 16S rRNA sequencing analysis with one or more bacteria selected from the group consisting of Porphyromonas gingivalis, Tannerella forsythia, Prevotella intermedia, Porphyromonas endodontalis, Filifactor alocis, Treponema denticola, and Rothia dentocariosa in a sample isolated from an individual; and comparing a bacterial % obtained through the 16S rRNA sequencing analysis with that selected from the group consisting of normal individuals and patients with gingivitis, moderate periodontitis, and severe periodontitis.

In addition, the present disclosure provides a composition for diagnosing a periodontal disease, including any one or more primer sets selected from the group consisting of a primer set represented by SEQ ID NOS: 1 and 2, a primer set represented by SEQ ID NOS: 3 and 4, a primer set represented by SEQ ID NOS: 5 and 6, a primer set represented by SEQ ID NOS: 7 and 8, a primer set represented by SEQ ID NOS: 9 and 10, a primer set represented by SEQ ID NOS: 11 and 12, and a primer set represented by SEQ ID NOS: 17 and 18.

In addition, the present disclosure provides a periodontal disease diagnostic kit including the composition.

### Advantageous Effects

The present disclosure seeks to distinguish the severity of a periodontal disease using colonies and a relative ratio of bacteria distributed in gingival crevicular fluid. Since differences are shown in characteristics of bacterial colonies that vary by gingivitis, moderate periodontitis, and severe periodontitis, presented are the criteria to distinguish the severity of periodontal diseases using the same. Moreover, when collecting samples, the gingival crevicular fluid has the advantage of being safe, easy, and fast to access while minimizing discomfort in patients owing to non-invasiveness, and thus the development of diagnostic kits using the gingival crevicular fluid may help grasp a degree of progression of the periodontal disease.

### BRIEF DESCRIPTION OF DRAWINGS

FIG. 1 shows results of comparing the diversity of colonies in each group via 16s rRNA sequencing analysis.

FIG. 2 shows a result of comparing colonies at a phylum level in each group via 16s rRNA sequencing analysis.

FIG. 3 shows a result of comparing colonies at a genera level in each group via 16s rRNA sequencing analysis.

FIG. 4 shows a result of comparing colonies at a species level in each group via 16s rRNA sequencing analysis.

FIG. 5 shows results of comparing the % of specific bacteria in each group via quantitative analysis by real-time PCR.

FIG. 6 shows results of comparing the count of specific bacteria in each group via quantitative analysis by real-time PCR.

FIG. 7 shows results of comparing a correlation between distribution of specific bacteria (16s rRNA sequencing %) and the sum of probing pocket depth in the whole oral cavity.

FIG. 8 shows results of comparing a correlation between distribution of specific bacteria (real-time PCR %) and the sum of probing pocket depth in the whole oral cavity.

FIG. 9 shows results of comparing a correlation between distribution of specific bacteria (real-time PCR count) and the sum of probing pocket depth in the whole oral cavity.

FIG. 10 shows results of comparing distribution of specific bacteria by a correlation between 16s rRNA sequencing % and real-time PCR % (spearman's correlation coefficient).

### BEST MODE FOR CARRYING OUT THE INVENTION

Hereinafter, the present disclosure will be described in more detail.

The term “periodontal disease” as used herein may include diseases that occur in periodontal tissues, including gingival (gum), periodontal ligaments, and bone tissues around the teeth, such as periodontitis and gingivitis. Depending on the severity of the disease, it is divided into gingivitis, moderate periodontitis, and severe periodontitis. It is known that, in the onset of the periodontal disease, the probing pocket is formed as the inflammation progresses to cause tissue damage, and the probing pocket becomes deeper with the severity of the periodontitis to cause inflammation in the periodontal ligament as the probing pocket deepens, finally resulting in induction of bone loss.

The term “diagnosis” as used herein refers to identification of the presence or characteristics of a pathological condition. For the purpose of the present disclosure, the diagnosis is to determine whether the periodontal disease has developed, a degree of disease progression, or whether there is a risk thereby.

The term “sample” as used herein may be gingival crevicular fluid, a fluid component infiltrated from gingival connective tissue through a thin crevicular epithelium, preferably gingival crevicular fluid that may be taken from a subject in a non-invasive manner. In the process of collecting the gingival crevicular fluid, bacteria that act as a direct cause of periodontitis present under the subgingival space are also collected. In other words, it is ultimately a sample in which subgingival bacteria are included.

The term “primer” as used herein refers to a short nucleic acid sequence having a short free 3′ hydroxyl group, which is capable of forming base pairs with a complementary template and acting as a starting point for template strand replication. The primer may initiate DNA synthesis in the presence of a reagent (i.e., DNA polymerase or reverse transcriptase) and four different nucleoside triphosphates for polymerization at an appropriate buffer and temperature. PCR conditions as well as a length of sense and antisense primers may be appropriately selected according to the techniques known in the art.

In the present disclosure, the oligonucleotide used as a primer may also include a nucleotide analogue, e.g., phosphorothioate, alkyl phosphophothioate, or peptide nucleic acid, or may include an intercalating agent.

Accordingly, the present disclosure provides a method of providing information necessary for diagnosis of a periodontal disease, including performing quantitative analysis by real-time polymerase chain reaction (real-time PCR) with one or more bacteria selected from the group consisting of Porphyromonas gingivalis, Tannerella forsythia, Prevotella intermedia, Porphyromonas endodontalis, Filifactor alocis, and Treponema denticola in a sample isolated from an individual; and comparing a bacterial % or a bacterial count obtained through the quantitative analysis by real-time PCR with that selected from the group consisting of normal individuals and patients with gingivitis, moderate periodontitis, and severe periodontitis.

In addition, the present disclosure provides a method of providing information necessary for diagnosis of a periodontal disease, including performing quantitative analysis by real-time polymerase chain reaction (real-time PCR) with one or more bacteria selected from the group consisting of Porphyromonas gingivalis, Tannerella forsythia, Prevotella intermedia, Porphyromonas endodontalis, Filifactor alocis, Treponema denticola, and Rothia dentocariosa in a sample isolated from an individual; and comparing a bacterial % or a bacterial count obtained through the quantitative analysis by real-time PCR with a set cut-off value indicating the presence of a probing pocket greater than or equal to 5 mm that shows the severity of periodontitis.

The sample may be gingival crevicular fluid.

The periodontal disease may be selected from the group consisting of, but is not limited to, severe periodontitis and recurrent periodontitis, and the periodontal disease may have a probing pocket depth greater than or equal to 5 mm.

The primer set may be, but is not limited to, a primer set for real-time polymerase chain reaction (real-time PCR).

The quantitative analysis by real-time polymerase chain reaction (real-time PCR) may measure an expression level of a target gene using any one or more primer sets selected from the group consisting of a primer set represented by SEQ ID NOS: 1 and 2, a primer set represented by SEQ ID NOS: 3 and 4, a primer set represented by SEQ ID NOS: 5 and 6, a primer set represented by SEQ ID NOS: 7 and 8, a primer set represented by SEQ ID NOS: 9 and 10, and a primer set represented by SEQ ID NOS: 11 and 12.

The target gene of the primer set represented by SEQ ID NOS: 1 and 2 may be rpoB in Porphyromonas gingivalis, the target gene of the primer set represented by SEQ ID NOS: 3 and 4 may be rpoB in Tannerella forsythia, the target gene of the primer set represented by SEQ ID NOS: 5 and 6 may be rpoB in Prevotella intermedia, the target gene of the primer set represented by SEQ ID NOS: 7 and 8 may be rpoB in Porphyromonas endodontalis, the target gene of the primer set represented by SEQ ID NOS: 9 and 10 may be 16s rRNA in Filifactor alocis, and the target gene of the primer set represented by SEQ ID NOS: 11 and 12 may be rpoB in Treponema denticola.

The comparing may determine, based on a cut-off value of the bacterial % obtained through the quantitative analysis by real-time PCR, a tooth having a probing pocket with a probing pocket depth greater than or equal to 5 mm based on the cut-off value, if the cut-off value of the target gene of the primer set represented by SEQ ID NOS: 1 and 2 is greater than or equal to 0.734, the cut-off value of the target gene of the primer set represented by SEQ ID NOS: 3 and 4 is greater than or equal to 0.009, the cut-off value of the target gene of the primer set represented by SEQ ID NOS: 5 and 6 is greater than or equal to 0.041, the cut-off value of the target gene of the primer set represented by SEQ ID NOS: 7 and 8 is greater than or equal to 0.048, the cut-off value of the target gene of the primer set represented by SEQ ID NOS: 9 and 10 is greater than or equal to 0.002, or the cut-off value of the target gene of the primer set represented by SEQ ID NOS: 11 and 12 is greater than or equal to 0.002. The criteria for determining the same are derived from the cut-off values of bacteria with an AUC value greater than or equal to 0.7 by obtaining the AUC value after drawing an ROC curve for diagnosing teeth with probing pockets greater than or equal to 5 mm based on the % of bacteria compared to the total bacteria collected from the tooth for the probing pocket of the tooth using samples of gingival crevicular fluid from a total of 51 people, including 7 teeth with healthy gums from 7 people with healthy gums, 14 teeth with gingivitis gums from 14 people with gingival gums, 12 teeth with moderate periodontitis from 12 people with moderate periodontitis, and 18 teeth with severe periodontitis from 18 people with severe periodontitis.

The comparing may determine, based on a cut-off value of the bacterial count obtained through the quantitative analysis by real-time PCR, a tooth having a probing pocket with a probing pocket depth greater than or equal to 5 mm based on the cut-off value, if the cut-off value of the target gene of the primer set represented by SEQ ID NOS: 1 and 2 is greater than or equal to 6489, the cut-off value of the target gene of the primer set represented by SEQ ID NOS: 3 and 4 is greater than or equal to 18, the cut-off value of the target gene of the primer set represented by SEQ ID NOS: 5 and 6 is greater than or equal to 125, the cut-off value of the target gene of the primer set represented by SEQ ID NOS: 7 and 8 is greater than or equal to 71, the cut-off value of the target gene of the primer set represented by SEQ ID NOS: 9 and 10 is greater than or equal to 3, or the cut-off value of the target gene of the primer set represented by SEQ ID NOS: 11 and 12 is greater than or equal to 23. The criteria for determining the same are derived from the cut-off values of bacteria with an AUC value greater than or equal to 0.7 by obtaining the AUC value after drawing an ROC curve for diagnosing teeth with probing pockets greater than or equal to 5 mm based on the count of bacteria collected from the teeth for the probing pocket of the tooth using samples of gingival crevicular fluid from a total of 51 people, including 7 teeth with healthy gums from 7 people with healthy gums, 14 teeth with gingivitis gums from 14 people with gingival gums, 12 teeth with moderate periodontitis from 12 people with moderate periodontitis, and 18 teeth with severe periodontitis from 18 people with severe periodontitis.

In addition, the present disclosure provides a method of providing information necessary for diagnosis of a periodontal disease, including performing 16S rRNA sequencing analysis with one or more bacteria selected from the group consisting of Porphyromonas gingivalis, Tannerella forsythia, Prevotella intermedia, Porphyromonas endodontalis, Filifactor alocis, Treponema denticola, and Rothia dentocariosa in a sample isolated from an individual; and comparing a bacterial % obtained through the 16S rRNA sequencing analysis with that selected from the group consisting of normal individuals and patients with gingivitis, moderate periodontitis, and severe periodontitis.

In addition, the present disclosure provides a method of providing information necessary for diagnosis of a periodontal disease, including performing 16S rRNA sequencing analysis with one or more bacteria selected from the group consisting of Porphyromonas gingivalis, Tannerella forsythia, Prevotella intermedia, Porphyromonas endodontalis, Filifactor alocis, Treponema denticola, and Rothia dentocariosa in a sample isolated from an individual; and comparing a bacterial % obtained through the 16S rRNA sequencing analysis with a set cut-off value indicating the presence of a probing pocket greater than or equal to 5 mm that shows the severity of periodontitis.

The cut-off values thereof are 0.170 for Porphyromonas gingivalis, 0.458 for Tannerella forsythia, 0.005 for Prevotella intermedia, 0.260 for Porphyromonas endodontalis, 0.145 for Filifactor alocis, 0.070 for Treponema denticola, and 0.240 for Rothia dentocariosa, and, in the case of Porphyromonas gingivalis, Tannerella forsythia, Prevotella intermedia, Porphyromonas endodontalis, Filifactor alocis, or Treponema denticola, a tooth may be determined to have a probing pocket greater than or equal to 5 mm when it is above the cut-off value, and in the case of Rothia dentocariosa, the tooth may be determined to have a probing pocket greater than or equal to 5 mm when it is below the cut-off value.

The sample may be gingival crevicular fluid.

The periodontal disease may be selected from the group consisting of, but is not limited to, severe periodontitis and recurrent periodontitis, and the periodontal disease may have a probing pocket depth greater than or equal to 5 mm.

The real-time polymerase chain reaction is a molecular biological polymerization method that amplifies a target using a target probe including a target primer and a label using cDNA as a template produced after reverse transcription of RNA into complementary DNA (cDNA) using reverse transcriptase, while quantitatively detecting a signal generated from the label of the target probe in the amplified target. This PCR method is well known in the art, and commercially available kits may also be used.

The PCR method may include analyzing a product amplified by PCR. The detection of the amplified product may be carried out by capillary electrophoresis, DNA chip, gel electrophoresis, radiometry, fluorimetry, or phosphorimetry. As one of the methods for detecting the amplified products, capillary electrophoresis may be performed. Capillary electrophoresis may be performed using, for example, an ABI sequencer. In addition, gel electrophoresis may be performed, in which agarose gel electrophoresis or acrylamide gel electrophoresis may be used depending on the size of the amplified product. In addition, in the fluorimetric method, PCR performed by labeling a 5′-end of the primer with Cy-5 or Cy-3 causes labeling with a fluorescent labelling material that detects the target sequence, and the fluorescence thus labeled may be measured using a fluorometer. In addition, in the radiometric method, radioisotopes such as ³²P or ³⁵S are added to a PCR reaction solution to label the amplified product during PCR, and then radioactivity may be measured using a radiometric instrument, e.g., a Geiger counter or a liquid scintillation counter.

The PCR method may include analyzing base sequences. Any of the methods known in the art may be used for the sequencing, specifically, but not limited to, automated sequencer may be used, or any one or more selected from among known methods may be used, such as pyrosequencing, restriction fragment length polymorphism (PCR-RELP), single strand conformation polymorphism (PCR-SSCP), specific sequence oligonucleotide (PCR-SSO), allele specific oligonucleotide (ASO) hybridization combined with a PCR-SSO method and dot hybridization, TaqMan-PCR, MALDI-TOF/MS, rolling circle amplification (RCA), high resolution melting (HRM), primer elongation, Southern blot hybridization, and dot hybridization.

In addition, the present disclosure provides a method of providing information necessary for diagnosis of a periodontal disease, including performing 16S rRNA sequencing analysis with one or more bacteria selected from the group consisting of Porphyromonas gingivalis, Tannerella forsythia, Treponema denticola, Prevotella intermedia, Porphyromonas endodontalis, Filifactor alocis, Fusobacterium nucleatum, Neisseria subflava, and Rothia dentocariosa in a sample isolated from an individual; and substituting a bacterial % obtained through the 16S rRNA sequencing analysis to Equations 1-1 to 1-4 below to calculate each value and determining gum disease severity corresponding to the Equation with the highest value thereamong as severity of the disease in a tooth:

$$Formula for healthy gums = - 6.64659 + ( 0.15264 ) \times ( P . ginivalis \% ) + ( - 0.14553 ) \times ( T . forsythia \% ) + ( - 0.53978 ) \times ( T . denticola \% ) + ( - 0.00038 ) \times ( P . intermedia \% ) + ( 0.10154 ) \times ( P . endodontalis \% ) + ( - 1.16269 ) \times ( F . alocis \% ) + ( 0.35602 ) \times ( F . nucleatum \% ) + ( 0.53214 ) \times ( R . dentocariosa \% ) + ( 0.49468 ) \times ( N . subflava \% ) \qquad [ Equation 1 - 1 ]$$

$$Formula for gums with gingivitis = - 2.92299 + ( 0.13318 ) \times ( P . ginivalis \% ) + ( - 0.295 ) \times ( T . forsythia \% ) + ( - 0.35928 ) \times ( T . denticola \% ) + ( 0.04931 ) \times ( P . intermedia \% ) + ( 0.09246 ) \times ( P . endodontalis \% ) + ( - 0.62252 ) \times ( F . alocis \% ) + ( 0.28945 ) \times ( F . nucleatum \% ) + ( 0.20002 ) \times ( R . dentocariosa \% ) + ( 0.22411 ) \times ( N . subflava \% ) \qquad [ Equation 1 - 2 ]$$

$$Formula for gums with monderate periodontitis = - 3.61834 + ( 0.00999 ) \times ( P . ginivalis \% ) + ( - 0.01267 ) \times ( T . forsythia \% ) + ( 0.61089 ) \times ( T . denticola \% ) + ( 0.15731 ) \times ( P . intermedia \% ) + ( 0.17845 ) \times ( P . endodontalis \% ) + ( - 0.40155 ) \times ( F . alocis \% ) + ( 0.23367 ) \times ( F . nucleatum \% ) + ( 0.16344 ) \times ( R . dentocariosa \% ) + ( 0.30398 ) \times ( N . subflava \% ) \qquad [ Equation 1 - 3 ]$$

$$Formula for gums with severe periodontitis = - 5.6885 + ( 0.33528 ) \times ( P . ginivalis \% ) + ( - 0.08898 ) \times ( T . forsythia \% ) + ( - 0.80137 ) \times ( T . denticola \% ) + ( 0.05997 ) \times ( P . intermedia \% ) + ( 0.21088 ) \times ( P . endodontalis \% ) + ( - 1.11862 ) \times ( F . alocis \% ) + ( 0.40108 ) \times ( F . nucleatum \% ) + ( 0.26583 ) \times ( R . dentocariosa \% ) + ( 0.33004 ) \times ( N . subflava \% ) \qquad [ Equation 1 - 4 ]$$

In addition, the present disclosure provides a method of providing information necessary for diagnosis of a periodontal disease, including performing quantitative analysis by real-time polymerase chain reaction (real-time PCR) with one or more bacteria selected from the group consisting of Porphyromonas gingivalis, Tannerella forsythia, Treponema denticola, Prevotella intermedia, Porphyromonas endodontalis, Filifactor alocis, Fusobacterium nucleatum, Neisseria subflava, and Rothia dentocariosa in a sample isolated from an individual; and substituting a bacterial % obtained through the quantitative analysis by real-time PCR to Equations 2-1 to 2-4 below to calculate each value and determining gum disease severity corresponding to the Equation with the highest value thereamong as severity of the disease in a tooth:

$$Formula for healthy gums = - 4.73715 + ( - 0.04148 ) \times ( P . ginivalis \% ) + ( 2.805198 ) \times ( T . forsythia \% ) + ( 2.18462 ) \times ( T . denticola \% ) + ( - 0.19053 ) \times ( P . intermedia \% ) + ( - 0.81998 ) \times ( P . endodontalis \% ) + ( 4.580745 ) \times ( F . alocis \% ) + ( 1.323154 ) \times ( F . nucleatum \% ) + ( 7.584086 ) \times ( R . dentocariosa \% ) + ( - 3.57656 ) \times ( N . subflava \% ) \qquad [ Equation 2 - 1 ]$$

$$Formula for gums with gingivitis = - 1.66994 + ( - 0.02906 ) \times ( P . ginivalis \% ) + ( 2.679294 ) \times ( T . forsythia \% ) + ( 1.969696 ) \times ( T . denticola \% ) + ( 0.2457071 ) \times ( P . intermedia \% ) + ( 0.025152 ) \times ( P . endodontalis \% ) + ( 5.319981 ) \times ( F . alocis \% ) + ( 0.480107 ) \times ( F . nucleatum \% ) + ( 1.29032 ) \times ( R . dentocariosa \% ) + ( 1.183753 ) \times ( N . subflava \% ) \qquad [ Equation 2 - 2 ]$$

$$Formula for gums with monderate periodontitis = - 4.57718 + ( - 0.1801 ) \times ( P . ginivalis \% ) + ( 15.36133 ) \times ( T . forsythia \% ) + ( 15.1877 ) \times ( T . denticola \% ) + ( 0.940892 ) \times ( P . intermedia \% ) + ( - 1.20232 ) \times ( P . endodontalis \% ) + ( 16.33778 ) \times ( F . alocis \% ) + ( 0.475167 ) \times ( F . nucleatum \% ) + ( - 0.52608 ) \times ( R . dentocariosa \% ) + ( 14.06681 ) \times ( N . subflava \% ) \qquad [ Equation 2 - 3 ]$$

$$Formula for gums with severe periodontitis = - 3.64997 + ( 0.225553 ) \times ( P . ginivalis \% ) + ( 0.750389 ) \times ( T . forsythia \% ) + ( - 0.09623 ) \times ( T . denticola \% ) + ( 0.265732 ) \times ( P . intermedia \% ) + ( - 0.03233 ) \times ( P . endodontalis \% ) + ( 18.2523 ) \times ( F . alocis \% ) + ( 0.526798 ) \times ( F . nucleatum \% ) + ( 0.5957 ) \times ( R . dentocariosa \% ) + ( 4.885258 ) \times ( N . subflava \% ) \qquad [ Equation 2 - 4 ]$$

In addition, the present disclosure provides a method of providing information necessary for diagnosis of a periodontal disease, including performing quantitative analysis by real-time polymerase chain reaction (real-time PCR) with one or more bacteria selected from the group consisting of Porphyromonas gingivalis, Tannerella forsythia, Treponema denticola, Prevotella intermedia, Porphyromonas endodontalis, Filifactor alocis, Fusobacterium nucleatum, Neisseria subflava, and Rothia dentocariosa in a sample isolated from an individual; and substituting a bacterial count obtained through the quantitative analysis by real-time PCR to Equations 3-1 to 3-4 below to calculate each value and determining gum disease severity corresponding to the Equation with the highest value thereamong as severity of the disease in a tooth:

$$Formula for healthy gums = - 2.09028 + ( 0.063613 ) \times ( P . ginivalis count ) + ( - 4.28627 ) \times ( T . forsythia count ) + ( - 2.29938 ) \times ( T . denticola count ) + ( 0.286028 ) \times ( P . intermedia count ) + ( - 2.59886 ) \times ( P . endodontalis count ) + ( - 1.76515 ) \times ( F . alocis count ) + ( 1.678029 ) \times ( F . nucleatum count ) + ( 63.93475 ) \times ( R . dentocariosa count ) + ( 45.44716 ) \times ( N . subflava count ) \qquad [ Equation 3 - 1 ]$$

$$Formula for gums with gingivitis = - 1.848 + ( 0.226929 ) \times ( P . ginivalis count ) + ( - 14.6669 ) \times ( T . forsythia count ) + ( - 10.1285 ) \times ( T . denticola count ) + ( 2.372836 ) \times ( P . intermedia count ) + ( - 9.03938 ) \times ( P . endodontalis count ) + ( - 8.53708 ) \times ( F . alocis count ) + ( 6.011738 ) \times ( F . nucleatum count ) + ( 103.446 ) \times ( R . dentocariosa count ) + ( 191.1352 ) \times ( N . subflava count ) \qquad [ Equation 3 - 2 ]$$

$$Formula for gums with monderate periodontitis = - 2.96588 + ( 0.17497 ) \times ( P . ginivalis count ) + ( - 6.25383 ) \times ( T . forsythia count ) + ( 0.027946 ) \times ( T . denticola count ) + ( 3.779165 ) \times ( P . intermedia count ) + ( - 7.31042 ) \times ( P . endodontalis count ) + ( - 8.88699 ) \times ( F . alocis count ) + ( 3.613806 ) \times ( F . nucleatum count ) + ( 34.77916 ) \times ( R . dentocariosa count ) + ( 400.9045 ) \times ( N . subflava count ) \qquad [ Equation 3 - 3 ]$$

$$Formula for gums with severe periodontitis = - 2.90144 + ( 0.682137 ) \times ( P . ginivalis count ) + ( - 45.8869 ) \times ( T . forsythia count ) + ( - 1.83537 ) \times ( T . denticola count ) + ( - 0.18553 ) \times ( P . intermedia count ) + ( 9.99311 ) \times ( P . endodontalis count ) + ( 3.639538 ) \times ( F . alocis count ) + ( 5.507213 ) \times ( F . nucleatum count ) + ( 92.53885 ) \times ( R . dentocariosa count ) + ( 203.7535 ) \times ( N . subflava count ) \qquad [ Equation 3 - 4 ]$$

In addition, the present disclosure provides a composition for diagnosing a periodontal disease, including any one or more primer sets selected from the group consisting of a primer set represented by SEQ ID NOS: 1 and 2, a primer set represented by SEQ ID NOS: 3 and 4, a primer set represented by SEQ ID NOS: 5 and 6, a primer set represented by SEQ ID NOS: 7 and 8, a primer set represented by SEQ ID NOS: 9 and 10, a primer set represented by SEQ ID NOS: 11 and 12, and a primer set represented by SEQ ID NOS: 17 and 18.

In addition, the present disclosure provides a periodontal disease diagnostic kit including the composition for diagnosing a periodontal disease.

The kit of the present disclosure may include, in addition to the primer set described above, conventional components included in a PCR kit. Conventional components included in the kit may be reaction buffers, polymerases, dNTPs (dATP, dCTP, dGTP, and dTTP), and cofactors such as Mg²⁺. A variety of DNA polymerases may be used in amplification steps, including the Klenow fragment of E. coli DNA polymerase I, thermostable DNA polymerase, and bacteriophage T7 DNA polymerase. Preferably, polymerase is a thermostable DNA polymerase that is obtainable from a variety of bacterial species. Most of the polymerases may be isolated from the bacteria themselves or commercially available.

### MODES FOR CARRYING OUT THE INVENTION

Hereinafter, the present disclosure will be described in more detail through example embodiments. These example embodiments are only for illustrating the present disclosure in more detail, and it will be apparent to those skilled in the art that the scope of the present disclosure is not limited by these example embodiments according to the gist of the present disclosure.

### Example 1: Selection of Study Subjects

Patients who came to the periodontal department of Ajou University Dental Hospital for examination, scaling, and periodontal treatment were recruited as study subjects. This study was conducted after receiving informed consent from all study subjects. Those who had orthodontic appliances in the oral cavity, other systemic medical history that may affect the progression of periodontitis, and a smoking habit, as well as those who had taken antibiotics and antimicrobial anti-inflammatory drugs in the three months prior to gingival crevicular fluid collection were excluded from the study subjects.

Periodontitis was diagnosed through probing pocket measurement performed before periodontal treatment. The classification of healthiness and disease severity in patients was based on the new classification criteria on the basis of the results of a conference jointly held by the European Society of Periodontology and the American Society of Periodontology in 2017 (J Periodontol. 2018 June; 89 Suppl 1: S159-S172.).

The definitions of healthy gums, gums with gingivitis, gums with moderate periodontitis, and gums with severe periodontitis are as follows.
\textcircled{1} Healthy gums: Subjects who have a probing pocket depth less than or equal to 3 mm and a bleeding on probing (BOP) index less than 10%
\textcircled{2} Gums with gingivitis: Subjects who have a probing pocket depth less than or equal to 3 mm and a BOP index greater than or equal to 10% on probing
\textcircled{3} Gums with moderate periodontitis: Subjects who have a probing pocket depth of 3-4 mm and a maximum probing pocket depth less than or equal to 5 mm and show mostly horizontal bone resorption without experiencing tooth extraction due to periodontitis (alveolar bone resorption is coronal third (15˜33%))
\textcircled{4} Gums with severe periodontitis: Subjects who have a probing pocket depth greater than or equal to 6 mm locally and show vertical bone resorption greater than or equal to 3 mm with posterior furcation-involved lesions (Class II or III)

In people belonging to these disease categories, bacteria from the area below the gums around the teeth showing the following characteristics that may characterize each group were collected and analyzed. Subgingival bacteria were collected from teeth with healthy gums of people with healthy gums, teeth with gingivitis gums of people with gingivitis, teeth with characteristics of moderate periodontitis of patients with moderate periodontitis, and teeth with characteristics of severe periodontitis of people with severe periodontitis, and the principles for selecting each subject and target tooth are as follows.
i) Healthy group: A case with probing pocket probe depth less than or equal to 3 mm with bleeding on probing in no more than 2 of the 6 regions of the tooth.
ii) Gingivitis group: A case with probing pocket probe depth less than or equal to 3 mm with bleeding on probing in no more than 3 of the 6 regions of the tooth.
iii) Moderate periodontitis group: A case having a region with a probing pocket probe depth of 4˜5 mm with bleeding on probing in 4 or more regions.
iv) Severe periodontitis group: A case having a region with a probing pocket probe depth greater than or equal to 6 mm with bleeding on probing in 4 or more regions.

### Example 2: Evaluation of Clinical Indicators

### 1) Clinical Indicators

The clinical indicators for periodontitis of the study subjects were evaluated by the plaque index for the whole oral cavity, gingival index, probing pocket depth, clinical attachment level, modified sulcus bleeding index, and degree of bleeding on probing.

### \textcircled{1} Plaque Index (PI): A Degree of Bacterial Film Deposition on a Surface of the Tooth

Score 0=No plaque
Score 1=Plaque that is scratched by a probe
Score 2=Obvious plaque visible to the eye
Score 3=Abundant plaque

### \textcircled{2} Gingival Index (GI): A Degree of Inflammation in the Gingiva

Score 0=No inflammation
Score 1=Mild inflammation accompanied by changes in the color and appearance in the gingiva, but no bleeding on probing
Score 2=Moderate inflammation accompanied by changes in the color and appearance in the gingiva with bleeding on probing
Score 3=Severe inflammation with natural abundant bleeding

### \textcircled{3} Probing Pocket Depth (PD)

It is a value obtained by measuring, using a periodontal probe (Hu-Friedy PCP 10, USA), a length from the gingival margin to the base of the probing pocket for a total of 6 parts (buccal mesial surface, buccal center, buccal distal surface, lingual mesial surface, lingual center, lingual distal surface), 3 parts each from the outside and inside of one tooth, meaning that the higher the value, the more severe the edema, alveolar bone destruction, and periodontitis.

### \textcircled{4} Clinical Attachment Level (CAL)

It is a value obtained by measuring, using a periodontal probe, a length from the cementoenamel junction to the base of the probing pocket for a total of 6 parts, 3 parts each from the outside and inside of one tooth and also a value obtained by adding the probing pocket depth with the amount of periodontal recession if there is a gingival recession, meaning that the higher the value, the more severe the periodontitis, just like the meaning of probing pocket depth.

\textcircled{5} Modified Sulcus Bleeding Index (mSBI)

On the basis of conventional methods (Mombelli et al. 1987), it is a value to evaluate the bleeding index within the modified sulcus using a periodontal probe for a total of 6 parts, 3 parts each from the outside and inside of one tooth, meaning that the higher the value, the more severe the periodontitis.
Score 0=A case with no bleeding
Score 1=A case with petechial hemorrhage
Score 2=A case in which bleeding is widespread along the gingival margin
Score 3=A case with abundant bleeding that oozes out

### \textcircled{6} Bleeding On Probing (BOP)

It is a value that shows an average of all teeth and is derived by recording the bleeding using a periodontal probe for a total of 6 parts, 3 parts each from the outside and inside of one tooth and then obtaining the percentage of spots where the bleeding is detected, meaning that the higher the value, the more severe the inflammation in the gingiva of the mouth.

### 2) Evaluation of Clinical Indicators

As shown in Table 1 below, the study subjects were divided into four groups and evaluated for clinical indicators. Evaluation was conducted on 7 people with healthy gums, 14 in the gingivitis group, 12 in the moderate periodontitis group, and 18 in the severe periodontitis group, and significant differences were found in the clinical indicators presented by each group.

TABLE 1
Characteristics of the clinical index of the teeth from which the whole oral
dentition and gingival crevicular fluid (GCF) of the subject were collected

| | | | | Moderate | Severe | | |
|---|---|---|---|---|---|---|---|
| | | Healthy gingiva | Gingivitis | periodontitis | periodontitis | | |
| | | (n = 7) | (n = 14) | (n = 12) | (n = 18) | P | pReg |
| Clinical index | Probing pocket (mm) | 1.90 ± 0.14 | 2.28 ± 0.26 | 2.74 ± 0.09 | 3.67 ± 0.57 | <.001 | <.001 |
| for whole oral | Clinical attachment level (mm) | 2.24 ± 0.28 | 2.36 ± 0.24 | 2.84 ± 0.15 | 4.25 ± 0.94 | <.001 | <.001 |
| dentition | Bleeding on probing (%) | 18.10 ± 4.15 | 51.25 ± 21.17 | 67.18 ± 14.13 | 74.00 ± 16.57 | <.001 | <.001 |
| | Plaque index | 0.21 ± 0.09 | 0.62 ± 0.43 | 1.24 ± 0.36 | 1.14 ± 0.46 | <.001 | <.001 |
| | Gingival index | 1.46 ± 0.08 | 1.82 ± 0.27 | 2.05 ± 0.21 | 2.17 ± 0.29 | <.001 | <.001 |
| | mSBI | 0.18 ± 0.04 | 0.67 ± 0.38 | 1.00 ± 0.33 | 1.20 ± 0.44 | <.001 | <.001 |
| Clinical index | Probing pocket (mm) | 2.10 ± 0.21 | 2.50 ± 0.25 | 3.22 ± 0.26 | 4.55 ± 0.80 | <.001 | <.001 |
| for sampled | Clinical attachment level (mm) | 2.38 ± 0.59 | 2.69 ± 0.48 | 3.26 ± 0.34 | 5.45 ± 1.21 | <.001 | <.001 |
| teeth | Plaque index | 0.00 ± 0.00 | 0.71 ± 0.69 | 146 ± 0.54 | 1.17 ± 0.45 | <.001 | <.001 |
| | Gingival index | 1.57 ± 0.45 | 2.04 ± 0.37 | 2.29 ± 0.40 | 2.39 ± 0.27 | 0.001 | <.001 |
| | mSBI | 0.20 ± 0.15 | 0.95 ± 0.46 | 1.58 ± 0.52 | 1.55 ± 0.49 | <.001 | <.001 |

mSB1; Modified sulcus bleeding index

### Example 3: Collection of Gingival Crevicular Fluid

All study subjects were advised to refrain from eating, brushing, and rinsing their mouths (mouthwash) for one hour before collection of the gingival crevicular fluid. In order to prevent contamination by saliva, gingival crevicular fluid was collected from the buccal mesial and buccal distal in one of first premolar and second premolar in the maxilla and first molar (two teeth in total) in the maxilla. If there is a dental plaque film at the site of gingival crevicular fluid collection, the supragingival dental plaque film was removed to prevent bleeding, and after the saliva on the tooth surface was excluded, a special test paper strip (Perio paper) was placed on the border between the tooth and the gingiva and inserted into the probing pocket. After leaving in the probing pocket for 30 seconds, the paper was put in a microtube including 100 μL of Dulbecco's Phosphate Buffered Saline (DPBS) and stored at −80° C. until analysis.

### Example 4: Next Generation Sequencing (16s rRNA Sequencing)

DNA extraction and sequencing (16S rRNA sequencing) of microtubes including the special paper test strip from which gingival crevicular fluid was collected were performed by ChunLab, Inc. After DNA extraction, the V3 and V4 regions were amplified using primers targeting the V3 and V4 regions of the bacterial 16S rRNA, and the PCR products were analyzed by the Illumina MiSeq sequencing system.

Using EzBioCloud (https://www.ezbiocloud.net/apps), a web-based analysis platform provided by ChunLab, Inc., the taxonomic profiles of bacterial colonies were analyzed and compared in the healthy, gingivitis, moderate periodontitis, and severe periodontitis groups. The Wilcoxon rank-sum test was used to compare the number of Operational Taxonomic Units (OTUs), Chaol index, Shannon index, and phylogenetic diversity for each group. In addition, the Kruskal-Wallis H test was performed to assess the difference in the predominant OTU in each group. OTU is a taxonomic unit that groups similar sequences by species from DNA sequencing results, and the Chaol and Shannon indices are indicators that assess the abundance and uniformity of colony, respectively.

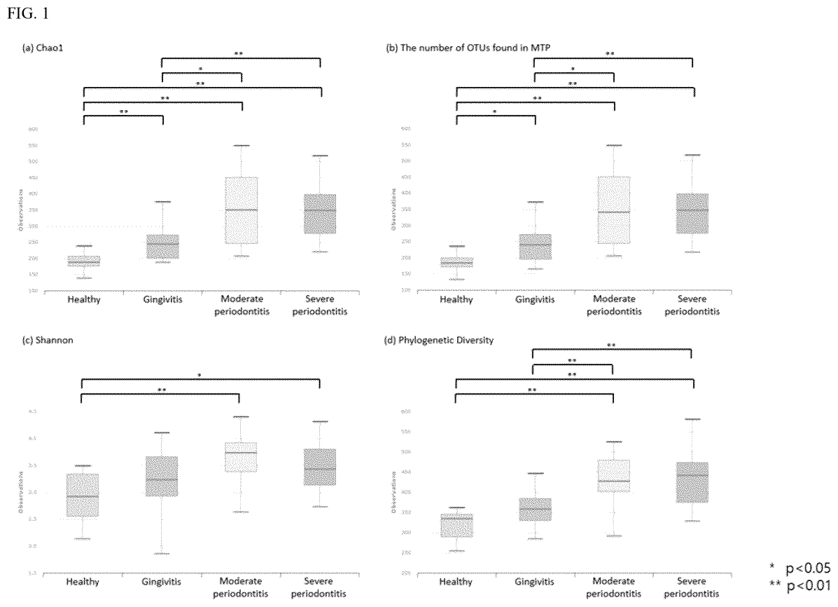

FIG. 1 shows a result of identifying a difference in the diversity in each group via 16s rRNA sequencing analysis. When it comes to diversity analysis, there are three main types of analysis (alpha diversity, beta diversity, and gamma diversity), of which alpha diversity is one that analyzes the distribution of various microorganisms present in a sample, referring to the average species diversity in a local-scale place or habitat. Presented Chao1, the number of OTUs found in MTP, Shannon, and phylogenetic diversity are all items that show alpha diversity. As shown in the results, it was found that the diversity increases with the severity of the disease, and various types of bacteria inhabit in the disease group than the healthy group.

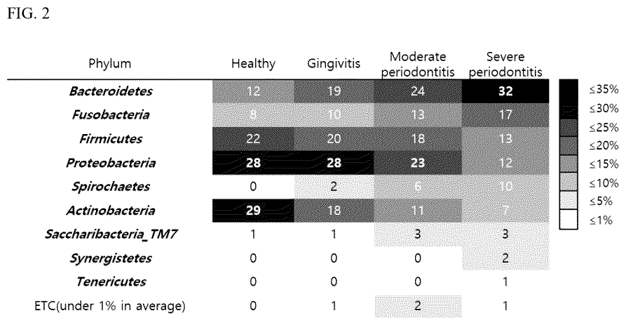

FIG. 2 shows a result of comparing colonies at a phylum level in each group via 16s rRNA sequencing analysis. Bacteroidetes, Fusobacteria, and Spirochaetes were found to increase significantly with the severity of the disease, while Actinobacteria was present in high concentrations in healthy gums and decreased significantly as the severity of the disease increased. In addition, high concentrations of Firmicutes and Proteobacteria were present in gingival crevicular fluid, with a tendency to decrease as the severity of the disease increased.

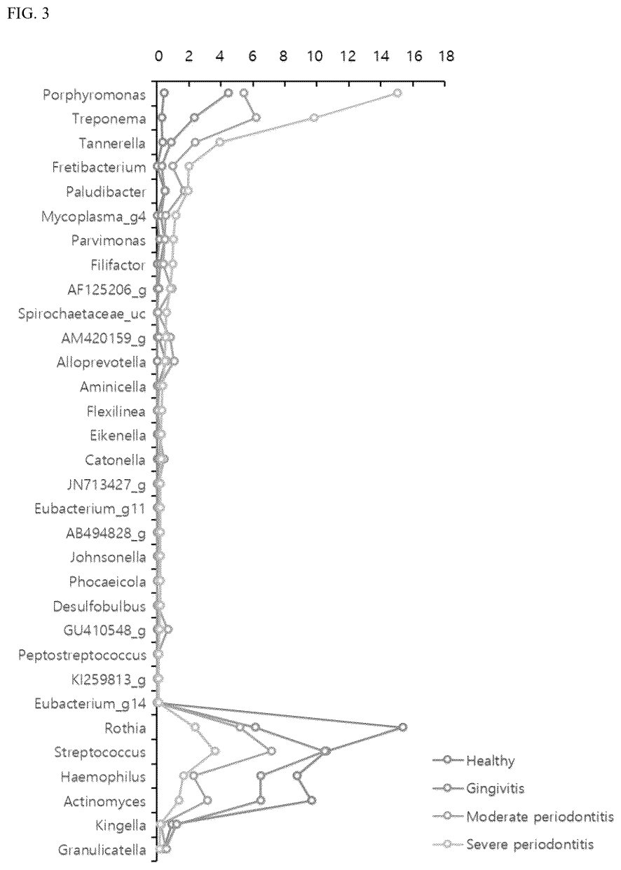

FIG. 3 shows a result of comparing colonies at a genera level in each group via 16s rRNA sequencing analysis. 25 genera including Porphyromonas, Treponema, and Tannerella were present in high proportions in the disease group, and 6 genera, including Rothia, Haemophilus, and Actinomyces, were present in high proportions in the healthy group.

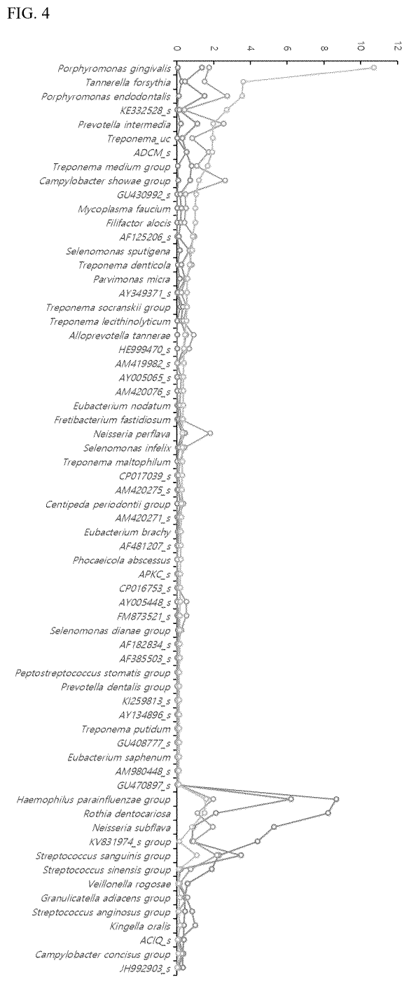

FIG. 4 shows a result of comparing colonies at a species level in each group via 16s rRNA sequencing analysis. 51 species, including Porphyromonas gingivalis, were present in high proportion in the disease group, and 13 species, including Haemophilus parainfluenzae, were present in high proportion in the healthy group. At the species level, it is indicated that bacterial species labeled as a group at the end of their names may include similar 16S rRNA bacterial species other than that species. The 16S rRNA sequencing technique reads only a part of the total sequence and determines the similarity to the base sequence in the published database by 97%, so it is sometimes determined to be identical to a bacterium other than a specific bacterium, in which case the most representative bacterial species are written with a group at the end of the name. For example, the F. nucleatum group may include all the following bacteria: F. nucleatum subspecies nucleatum, vincentii, polymorphum, fusiforme, animalis, F. simiae, F. canifelinum, F. hwasookii.

### Example 5: Quantitative Analysis of Specific Bacteria Using Real-Time (RT) PCR

Based on the results of 16S rRNA sequencing, the following 9 types of bacteria were selected that showed statistical significance for each group, and quantitative analysis was performed by real-time PCR using a species-specific primer:
Porphyromonas gingivalis, Tannerella forsythia, Treponema denticola, Prevotella intermedia, Porphyromonas endodontalis, Filifactor alocis, Fusobacterium nucleatum, Neisseria subflava, Rothia dentocariosa

DNA extraction of gingival crevicular fluid placed and stored in 100 μL of DPBS was performed by ChunLab, Inc. The total bacterial count was analyzed via real-time PCR using a universal primer (FEMS Immunol Med Microbiol. 2003 Oct. 24; 39 (1): 81-6.) targeting 16S rRNA. Considering the copy number of 16s rRNA in a single bacterium, the result value of real-time PCR was divided by 6 and considered as a single bacterium.

Of the 9 species of bacteria, the bacterial counts were calculated on the basis of formulas described in the paper (J Microbiol. 2011 April; 49 (2): 315-9.) for the bacterium P. gingivalis, the paper (International Journal of Oral Biology Vol. 40 No. 4 pp. 205-210) for bacterium P. intermedia, and the paper (Arch Microbiol. 2013 July; 195 (7): 473-82.) for 6 species of bacteria (T. forsythia, T. denticola, P. endodontalis, F. nuclatum, R. dentocariosa, N. subflava). For the other bacterium F. alocis, in reference to the paper (Sci Rep. 2016 Sep. 19; 6:33638.), the DNA of the actual bacteria was extracted, the standard curve was set by performing real-time PCR for each concentration, and thereby the real-time PCR of the gingival crevicular fluid sample to be identified was performed by obtaining the Equation (first-order equation) for the real-time PCR value related to the bacterial concentration, followed by quantitative analysis on the bacteria in the sample based on this Equation. The Equations were obtained as the average value of the same experiment repeated three times independently.

Each real-time PCR was performed in a total volume of 20 μL, including 2 μL of genomic DNA, 2 μL of forward and reverse primers, 6 μL of sterile DNase-RNase-free water, and 10 μL of TOP Real™ qPCR 2× PreMIX. All data were analyzed using TOP Real™ qPCR 2× PreMIX (SYBR Green) kit (Enzynomics, Daejeon, Korea) and Exicycler™ 96 V4 Real-Time Quantitative Thermal Block (Bioneer, Daejeon, Korea). The sequences of the primer used in the experiment are shown in Table 2 below.

TABLE 2

| | Primer | | Sequence |
|---|---|---|---|
| Bacteria | Target | Sequence (5′ to 3′) | Number |
| P. gingivalis | rpoB | Pg-F: GGA AGA GAA GAC CGT AGC ACA | 1 |
| | | AGG A | |
| | | Pg-R: GAG TAG GCG AAA CGT CCA TCA | 2 |
| | | GGT C | |
| T. forsythia | rpoB | Tf-F2: GGATTGACCACCGGCGAAGACA | 3 |
| | | Tf-R2: | 4 |
| | | CGGACACGACGGTTACTCAAATGG | |
| P. intermedia | rpoB | RTPi-F: ACC CAT TGG CAG AAG TTA CG | 5 |
| | | RTPi-R: TCA ATA GGA CAC AAG CGA | 6 |
| | | CCA TAG | |
| P. | rpoB | Pe-F1: | 7 |
| endodontalis | | AGCAGAGTTCCGTCGTCGTATTCA | |
| | | Pe-R1: GCGCCCTGCCATCTTGTCTC | 8 |
| F. alocis | 16S rRNA | F: AACCGGAGCAAAACTGAGAA | 9 |
| | | R: CCGTCCGCCACTAACTTCTA | 10 |
| T. denticola | rpoB | Td-F2: CGGGCGTGCATCTTGTCGTCTAC | 11 |
| | | Td-R2: CTTAACCGGCCGCCTCTTTGAA | 12 |
| F. nucleatum | rpoB | Fn-F1: ACCTAAGGGAGAAACAGAACCA | 13 |
| | | Fn-R1: CCTGCCTTTAATTCATCTCCAT | 14 |
| N. subflava | rpoB | Nsub-F6: TTGGCTCAATTGGCTGGAA | 15 |
| | | Nsub-R6: | 16 |
| | | GTAATCGTCGTGTTCGTTCTGTG | |
| R. | rpoB | Rd-F8: CTGGGCAAAGCGTCTGGAAAAC | 17 |
| dentocariosa | | Rd-R8: | 18 |
| | | GAAATCACGAATCGGGGAAATCTC | |
| Universal | 16S rRNA | F: GTG STG CAY GGY TGT CGT CA | 19 |
| primer | | R: ACG TCR TCC MCA CCT TCC TC | 20 |

### Example 6: Statistical Analysis

The Kruskal-Wallis H test was performed to analyze whether the results of real-time PCR for each of the 9 bacteria showed differences in each group. Spearman's correlation coefficient analysis was performed to determine the correlation between 16s rRNA sequencing (%) and real-time PCR (%) in specific bacteria.

Probing pockets were measured for the entire tooth by 6 parts per tooth, and the sum thereof was evaluated by simple linear regression analysis for the correlation between the sum of the probing pocket depth and the concentration of specific bacteria (16s rRNA sequencing %, real-time PCR %, real-time PCR count) as a basis that may reflect the severity of periodontitis in the entire dentition (oral cavity) (%: a ratio of the bacteria to the total bacteria, count: an amount of bacteria corresponding to approximately 10% of the total bacteria collected with the paper after placing Perio paper on 2 sites of the buccal mesial and buccal distal of a single tooth).

To obtain the optimal cut-off value for each bacterium that distinguishes the severity of the disease, Receiver Operating Characteristic (ROC) curves were drawn. The cut-off value for each bacterium that distinguishes the probing pocket depth greater than or equal to 5 mm that shows the severity of periodontitis was obtained, the cut-off value was presented for the value with the highest Area under the ROC Curve (AUC), and the sensitivity and specificity were presented.

### 1) Quantitative Analysis Results of Specific Bacteria by Real-Time PCR

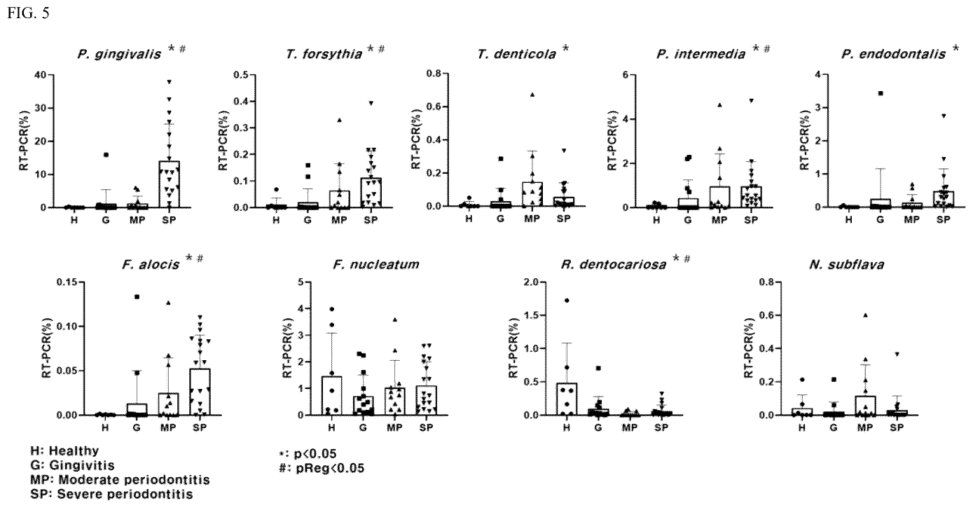

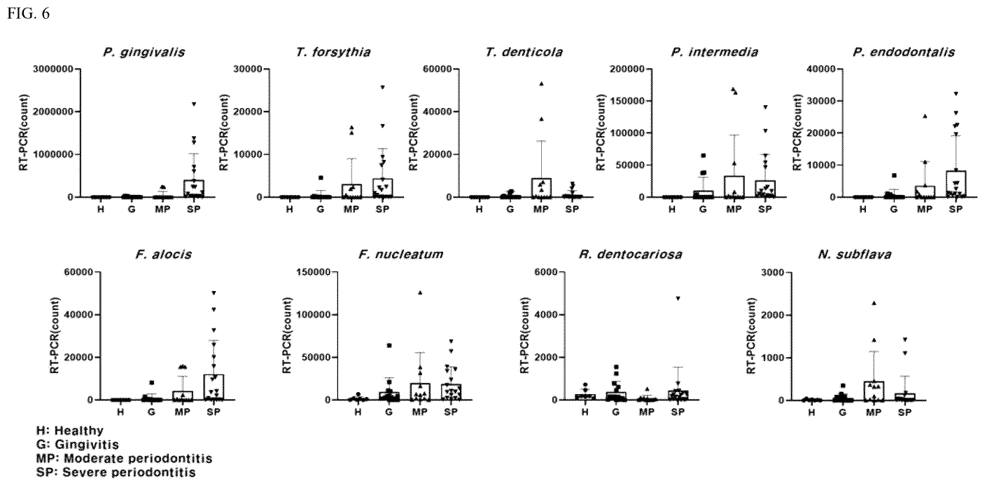

FIGS. 5 and 6 show results of comparing the % and count of specific bacteria in each group based on the quantitative analysis result by real-time PCR of specific bacteria. The graph values in FIGS. 4 and 5 are shown in Table 3 and Table 4 below, respectively.

In regard to the % result, it was found that P. gingivalis, T. forsythia, T. denticola, P. intermedia, P. endodontalis, and F. alocis were present in high concentrations in severe periodontitis, and R. dentocariosa was present in high concentrations in the healthy group. In regard to the count results, it was found that the remaining bacteria, except R. dentocariosa, were present in significantly higher concentrations in gingival crevicular fluid as the severity of the disease increased.

For reference, pReg represents p-value of whether the mean increases or decreases, given with values of 0, 1, 2, and 3 from the healthy group to the severe periodontitis group, respectively. In other words, significance in p-value means that there is a difference between groups, and significance in pReg means that there is a tendency for the value to increase or decrease as it progresses to the healthy, gingivitis, moderate periodontitis, and severe periodontitis groups.

TABLE 3

| | Healthy | | Moderate | Severe | | |
|---|---|---|---|---|---|---|
| | Gingivitis | Gingivitis | Periodontitis | periodontitis | | |
| var | (Healthy) | (Gingivitis) | (Moderate) | (Severe) | p | pReg |
| P. gingivalis | 0.0560 ± 0.1180 | 1.2290 ± 4.2330 | 1.2760 ± 2.1660 | 14.0660 ± 11.1110 | <.001 | <.001 |
| T. forsythia | 0.0110 ± 0.0260 | 0.0200 ± 0.0510 | 0.0650 ± 0.1010 | 0.1130 ± 0.1000 | <.001 | 0.001 |
| T. denticola | 0.0090 ± 0.0190 | 0.0300 ± 0.0780 | 0.1470 ± 0.1870 | 0.0570 ± 0.0840 | <.001 | 0.223 |
| P. intermedia | 0.0580 ± 0.1010 | 0.4340 ± 0.8180 | 0.9620 ± 1.4750 | 0.9600 ± 1.1160 | <.001 | 0.036 |
| P. | 0.0090 ± 0.0210 | 0.2510 ± 0.9150 | 0.1360 ± 0.2440 | 0.4880 ± 0.6730 | <.001 | 0.109 |
| endodontalis | | | | | | |
| F. alocis | 0.0000 ± 0.0010 | 0.0130 ± 0.0370 | 0.0250 ± 0.0400 | 0.0530 ± 0.0380 | <.001 | <.001 |
| F. nucleatum | 1.4750 ± 1.6120 | 0.7190 ± 0.7890 | 1.0360 ± 1.0240 | 1.1130 ± 0.8900 | 0.448 | 0.968 |
| R. | 0.4840 ± 0.5990 | 0.0950 ± 0.1860 | 0.0210 ± 0.0330 | 0.0570 ± 0.0930 | 0.009 | 0.004 |
| dentocariosa | | | | | | |
| N. subflava | 0.0420 ± 0.0790 | 0.0210 ± 0.0580 | 0.1160 ± 0.1870 | 0.0300 ± 0.0860 | 0.344 | 0.864 |

TABLE 4

| var | total | Healthy | Gingivitis |
|---|---|---|---|
| P. gingivalis | 154401.74 ± 407061.90 | 38.42 ± 76.48 | 4373.33 ± 10181.71 |
| T. forsythia | 2372.66 ± 5323.83 | 4.84 ± 10.63 | 355.75 ± 1209.36 |
| T. denticola | 2587.27 ± 8984.22 | 4.17 ± 8.11 | 398.72 ± 895.74 |
| P. intermedia | 20028.54 ± 41231.39 | 16.30 ± 35.38 | 10392.38 ± 20815.13 |
| P. endodontalis | 3927.35 ± 8093.40 | 4.33 ± 9.06 | 652.70 ± 1803.84 |
| F. alocis | 5478.66 ± 11148.18 | 1.61 ± 2.13 | 759.31 ± 2166.86 |
| F. nucleatum | 14050.94 ± 23239.05 | 1632.73 ± 2397.60 | 9257.78 ± 16758.05 |
| R. dentocarlosa | 319.36 ± 709.28 | 275.29 234.35 | 382.51 ± 494.00 |
| N. subflava | 182.27 ± 438.47 | 12.54 ± 15.91 | 61.78 ± 96.13 |

| var | Moderate | Severe | P | pReg |
|---|---|---|---|---|
| P. gingivalis | 42057.81 ± 94225.32 | 406016.63 ± 614195.27 | <.001 | 0.004 |
| T. forsythia | 3029.96 ± 6001.65 | 4423.98 ± 6965.47 | <.001 | 0.014 |
| T. denticola | 8935.86 ± 17374.39 | 1061.62 ± 1962.95 | <.001 | 0.571 |
| P. intermedia | 33256.95 ± 63847.53 | 26486.92 ± 40273.47 | <.001 | 0.091 |
| P. endodontalis | 3584.13 ± 7575.85 | 8228.75 ± 10871.49 | <.001 | 0.003 |
| F. alocis | 4212.95 ± 6967.69 | 12123.03 ± 15873.30 | <.001 | 0.001 |
| F. nucleatum | 19871.39 ± 35796.30 | 18727.96 ± 20322.18 | 0.022 | 0.066 |
| R. dentocarlosa | 78.27 ± 146.10 | 448.12 ± 1094.15 | 0.050 | 0.708 |
| N. subflava | 445.38 ± 704.02 | 166.59 ± 405.77 | 0.048 | 0.262 |

### 2) Results of Analyzing the Correlation Between Distribution of Specific Bacteria and the Sum of the Probing Pocket Depth

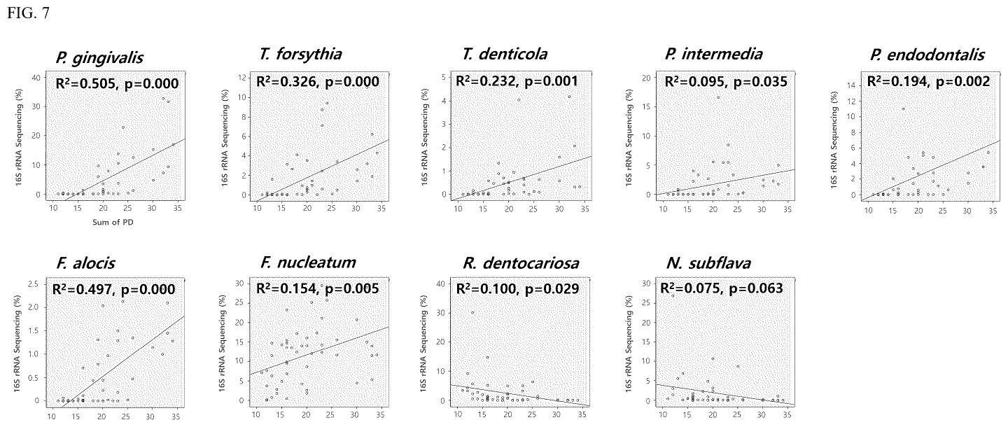

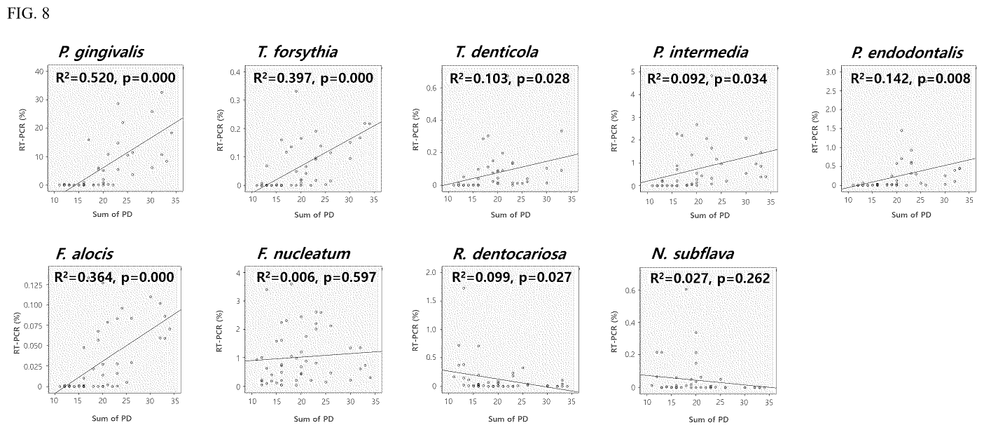

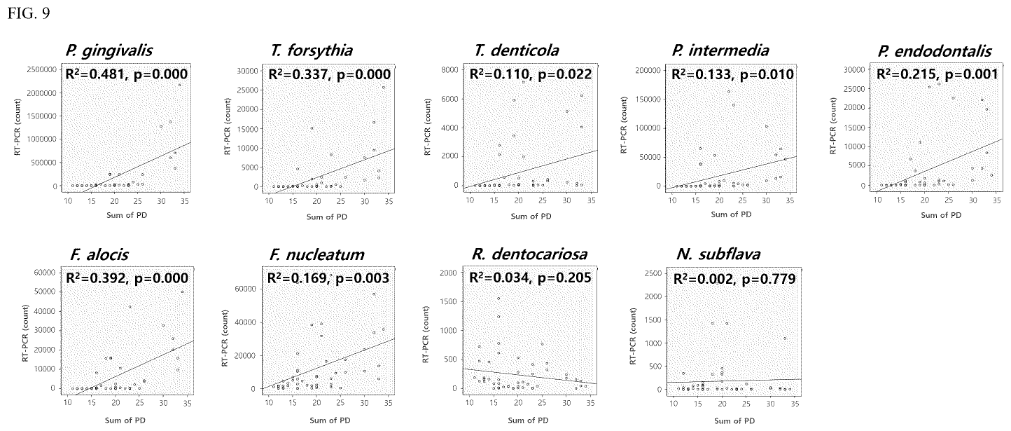

FIGS. 7 to 9 show analysis on the correlation between distribution of specific bacteria (16S rRNA sequencing (%) and the sum of real-time PCR (% or count) and the probing pocket depth in the whole oral cavity, and the values of each graph are shown in Table 5 below.

In analyzing the correlation with the distribution of specific bacteria, the sum of the probing pocket depth among several clinical indicators (PD, mSBI, BOP %, CAL, PI, GI, etc.) that distinguish the severity of diseases was selected because the deeper the probing pocket depth, the more dysbiosis of the subgingival flora was advanced, assuming that the result of the dysbiosis of these subgingival flora would be reflected in the gingival crevicular fluid. The table presented shows the correlation between the bacterial concentration and the sum of the probing pocket depth in the whole oral cavity, meaning that the higher the R2 value, the higher the correlation.

In the case of 16S rRNA sequencing (%), there was a negative correlation with N. subflava with no statistical significance, while a significant positive correlation was found in all remaining bacteria except R. dentocariosa and N. subflava. In other words, it may indicate that the deeper the overall probing pocket, the higher the concentration of the corresponding bacteria. R. dentocariosa showed a significant negative correlation, which may be said to reflect that the deeper the overall probing pocket depth, the lower the concentration of the bacteria.

For real-time PCR (%), F. nucleatum and N. subflava did not derive statistically significant results. R. dentocariosa showed a significant negative correlation, while a significant positive correlation was shown in the rest of the bacteria.

In the case of real-time PCR (count), a positive correlation was shown in all bacteria except R. dentocariosa and N. subflava.

TABLE 5

| | | P | T. | T. | F. | P. | P | F | R. | N. |
|---|---|---|---|---|---|---|---|---|---|---|
| | | gingivalia | forsythia | denticola | alocis | intermedia | endodontalis | nucleatum | dentocariosa | subflava |
| NGS | R2 | .505 | .326 | .232 | .095 | .194 | .497 | .154 | .100 | .075 |
| (%) | beta | .878 | .239 | .269 | .156 | .277 | .079 | .444 | −.256 | −.187 |
| | P- | .000 | .000 | .001 | .035 | .002 | .000 | .005 | .029 | .063 |
| | value | | | | | | | | | |
| RT-PCR | R2 | .520 | .397 | .103 | .092 | .142 | .364 | .006 | .099 | .027 |
| (%) | beta | 1.070 | .009 | .007 | .053 | .029 | .004 | .012 | −.014 | −.003 |
| | P- | .000 | .000 | .028 | .034 | .008 | .000 | .597 | .027034 | .262 |
| | value | | | | | | | | | |
| RT-PCR | R2 | .481 | .337 | .110 | .133 | .215 | .392 | .169 | .034 | .002 |
| (count) | beta | 46083.883 | 458.627 | 94.989 | 2046.009 | 516.406 | 1135..215 | 1095.391 | −9.431 | 2.689 |
| | P- | .000 | .000 | .022 | .010 | .001 | .000 | .003 | .205 | .779 |
| | value | | | | | | | | | |

3) Results of Analyzing the Correlation with 16S rRNA Sequencing (%) and Real-Time PCR (%) of Specific Bacteria

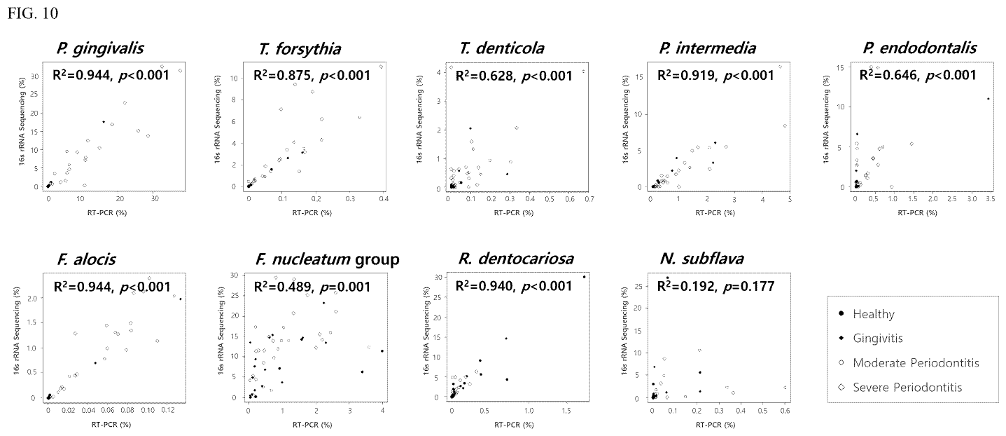

In the case of the 16S rRNA sequencing technique, if it reads only a part of the entire base sequence and shows a 97% similarity to the base sequence in the published database, it is determined to be the bacterium, such that it may be relatively lower in accuracy than real-time PCR. Thereby, the result of analyzing the correlation between the % value of the bacteria compared to the total bacteria obtained from the results of 16S rRNA sequencing (Next Generation Sequencing, NGS) and the % of the bacteria compared to the total bacteria obtained from the real-time PCR results is shown in FIG. 10.

As a result of analysis, it appeared that the remaining bacteria except N. subflava had a statistically significant correlation, especially P. gingivalis, F. alocis, R. dentocariosa, and P. intermedia had high significance, while F. nucleatum had relatively low significance. The results of 16S rRNA sequencing for F. nucleatum may be seen as a group to include all F. nucleatum subspecies nucleatum, vincentii, polymorphum, fusiforme, animalis, F. Simiae, F. canifelinum, and F. hwasooki. In the case of the species-specific primer of F. nucleatum used in real-time PCR also, the correlation between results from the two experimental methods may be low because it is not the primer for F. nucleatum alone, but rather a primer that detects both F. nucleatum and F. simiae.

### 4) Establishment of Reference Values for Each Bacterium to Distinguish the Severity of Diseases and Diagnosis Methods

(1) (Probing Pocket Depth Less than 5 mm) Vs. (Probing Pocket Depth Greater than or Equal to 5 mm)

Presence of the probing pocket depth greater than or equal to 5 mm may be recognized as one of the important risk factors for periodontitis recurrence as well as severe periodontitis. Collection of gingival crevicular fluid from teeth with the probing pocket depth greater than or equal to 5 mm may allow detection of bacterial colonies in areas with a high risk of periodontitis recurrence. In other words, this criterion means that it is possible to distinguish areas with a high risk of periodontitis recurrence, and bacteria with an AUC greater than or equal to 0.7 are meaningful as a diagnostic value.
\textcircled{1} Bacteria with AUC greater than or equal to 0.7
\textcircled{2} 16s rRNA sequencing (%): P. gingivalis, T. forsythia, T. denticola, P. intermedia, P. endodontalis, F. alocis, R. dentocariosa
\textcircled{3} RT-PCR (%): P. gingivalis, T. forsythia, T. denticola, P. intermedia, P. endodontalis, F. alocis
\textcircled{4} RT-PCR (count): P. gingivalis, T. forsythia, T. denticola, P. intermedia, P. endodontalis, F. alocis

### 6) Concentration of Bacteria as a Standard for Diagnosing Periodontitis Using Gingival Crevicular Fluid and a Diagnosis Method Thereof

In the case of P. gingivalis, when the concentration of the bacteria in the gingival crevicular fluid is measured by real-time PCR using the primer shown in Table 2, if the % of P. gingivalis compared to the total bacteria is less than or equal to 0.734%, it indicates that it is the relatively healthy gum that is more likely to show probing pockets less than equal to 5 mm, and if it is greater than 0.734%, it indicates that the depth of the probing pocket is likely to be greater than or equal to 5 mm. The AUC of P. gingivalis diagnosed as teeth with probing pockets greater than or equal to 5 mm is 0.863, wherein the criterion of 0.734% is a way to distinguish between teeth with gums with a low risk of recurrence for periodontitis less than 5 mm and teeth with gums with a high risk of recurrence for periodontitis greater than or equal to 5 mm. It has a sensitivity of 0.864 and a specificity of 0.862.

TABLE 6

| | | Cut-off | | | |
|---|---|---|---|---|---|
| | | value | AUC | Sensitivity | Specificity |
| NGS | P. gingivalis | 0.170 | 0.851 | 0.909 | 0.793 |
| | T. forsythia | 0.485 | 0.828 | 0.864 | 0.793 |
| | T. denticola | 0.070 | 0.759 | 0.864 | 0.655 |
| | P. intermedia | 0.005 | 0.810 | 1.000 | 0.621 |
| | P. endodontalis | 0.260 | 0.737 | 0.818 | 0.655 |
| | F. alocis | 0.145 | 0.760 | 0.727 | 0.793 |
| | F. nucleatum | 17.280 | 0.642 | 0.318 | 0.966 |
| | R. dentocariosa | 0.240 | 0.726 | 0.409 | 0.138 |
| | N. subflava | 0.075 | 0.687 | 0.455 | 0.172 |
| Real-time | P. gingivalis | 0.734 | 0.863 | 0.864 | 0.862 |
| PCR | T. forsythia | 0.009 | 0.851 | 0.909 | 0.793 |
| (%) | T. denticola | 0.002 | 0.725 | 0.864 | 0.586 |
| | P. intermedia | 0.041 | 0.810 | 1.000 | 0.621 |
| | P. endodontalis | 0.048 | 0.812 | 0.727 | 0.897 |
| | F. alocis | 0.002 | 0.788 | 0.818 | 0.759 |
| | F. nucleatum | 0.206 | 0.570 | 0.864 | 0.276 |
| | R. dentocariosa | 0.011 | 0.675 | 0.409 | 0.241 |
| | N. subflava | 0.001 | 0.595 | 0.500 | 0.310 |
| Real-time | P. gingivalis | 6489 | 0.857 | 0.818 | 0.897 |
| PCR | T. forsythia | 18 | 0.817 | 0.909 | 0.724 |
| (count) | T. denticola | 23 | 0.742 | 0.864 | 0.621 |
| | P. intermedia | 125 | 0.845 | 1.000 | 0.690 |
| | P. endodontalis | 71 | 0.834 | 0.909 | 0.759 |
| | F. alocis | 3 | 0.776 | 1.000 | 0.552 |
| | F. nucleatum | 8432 | 0.692 | 0.591 | 0.793 |
| | R. dentocariosa | 211 | 0.544 | 0.364 | 0.724 |
| | N. subflava | 3 | 0.610 | 0.909 | 0.310 |

7) A Method of Utilizing Gingival Crevicular Fluid Samples for the Disease Severity Based on Equations Obtained from 16S rRNA Sequencing Analysis and Real-Time PCR Analysis

Using the concentration of 9 species of bacteria in gingival crevicular fluid, the severity of the disease in the teeth from which the sample was collected may be distinguished into healthy gums, gingivitis, moderate periodontitis, and severe periodontitis.

1. Method of Utilizing Concentrations of 9 Species of Bacteria Obtained Through the Method of 16S rRNA Sequencing

There are four Formulas that yield healthy gums, gums with gingivitis, gums with moderate periodontitis, and gums with severe periodontitis according to the severity of the disease.

$$Formula for healthy gums = - 6.64659 + ( 0.15264 ) \times ( P . ginivalis \% ) + ( - 0.14553 ) \times ( T . forsythia \% ) + ( - 0.53978 ) \times ( T . denticola \% ) + ( - 0.00038 ) \times ( P . intermedia \% ) + ( 0.10154 ) \times ( P . endodontalis \% ) + ( - 1.16269 ) \times ( F . alocis \% ) + ( 0.35602 ) \times ( F . nucleatum \% ) + ( 0.53214 ) \times ( R . dentocariosa \% ) + ( 0.49468 ) \times ( N . subflava \% ) \qquad ( 1 )$$

$$Formula for gums with gingivitis = - 2.92299 + ( 0.13318 ) \times ( P . ginivalis \% ) + ( - 0.295 ) \times ( T . forsythia \% ) + ( - 0.35928 ) \times ( T . denticola \% ) + ( 0.04931 ) \times ( P . intermedia \% ) + ( 0.09246 ) \times ( P . endodontalis \% ) + ( - 0.62252 ) \times ( F . alocis \% ) + ( 0.28945 ) \times ( F . nucleatum \% ) + ( 0.20002 ) \times ( R . dentocariosa \% ) + ( 0.22411 ) \times ( N . subflava \% ) \qquad ( 2 )$$

$$Formula for gums with monderate periodontitis = - 3.61834 + ( 0.00999 ) \times ( P . ginivalis \% ) + ( - 0.01267 ) \times ( T . forsythia \% ) + ( 0.61089 ) \times ( T . denticola \% ) + ( 0.15731 ) \times ( P . intermedia \% ) + ( 0.17845 ) \times ( P . endodontalis \% ) + ( - 0.40155 ) \times ( F . alocis \% ) + ( 0.23367 ) \times ( F . nucleatum \% ) + ( 0.16344 ) \times ( R . dentocariosa \% ) + ( 0.30398 ) \times ( N . subflava \% ) \qquad ( 3 )$$

$$Formula for gums with severe periodontitis = - 5.6885 + ( 0.33528 ) \times ( P . ginivalis \% ) + ( - 0.08898 ) \times ( T . forsythia \% ) + ( - 0.80137 ) \times ( T . denticola \% ) + ( 0.05997 ) \times ( P . intermedia \% ) + ( 0.21088 ) \times ( P . endodontalis \% ) + ( - 1.11862 ) \times ( F . alocis \% ) + ( 0.40108 ) \times ( F . nucleatum \% ) + ( 0.26583 ) \times ( R . dentocariosa \% ) + ( 0.33004 ) \times ( N . subflava \% ) \qquad ( 4 )$$

After collecting the gingival crevicular fluid of teeth to know the severity of the disease and then performing 16S rRNA sequencing on the bacteria present therein, the concentration % of the bacteria relative to the total bacteria for the 9 bacterial species may be calculated and then the % of the 9 bacterial species calculated may be applied to 4 Formulas to determine the severity of gum disease corresponding to the Formula with the highest value as the severity of disease in the teeth.

When applying the % for each of the 9 bacterial species derived from 16S rRNA sequencing to corresponding Formula for the analyzed gingival crevicular fluid samples from the total of 51 people, including 7 teeth with healthy gums from 7 people with healthy gums, 14 teeth with gingival gums from 14 people with gingival gums, 12 teeth with moderate periodontitis from 12 people with moderate periodontitis, and 18 teeth with severe periodontitis from 18 people with severe periodontitis,

Of the 7 samples from healthy gums, 3 samples (42.86%) were analyzed as healthy gums, and 4 samples (57.14%) as gingivitis gums.

Of the 14 samples from gingivitis gums, 11 samples (78.57%) were analyzed as gingivitis gums, 1 sample (7.14%) as healthy gums, 1 sample (7.14%) as moderate periodontitis gum, and 1 sample (7.14%) as severe periodontitis gum.

Of the 12 samples from moderate periodontitis gums, 7 samples (58.33%) were analyzed as moderate periodontitis gums, and 5 samples (41.67%) as gingivitis gums.

Of the 18 samples from severe periodontitis gums, 12 samples (66.67%) were analyzed as severe periodontitis gums, 1 sample (5.56%) as healthy gums, 3 samples (16.67%) as gingivitis gums, and 2 samples (11.11%) as moderate periodontitis gums.

### 2. Method of Utilizing the % of the Bacteria Compared to the Total Bacteria of 9 Bacterial Species Obtained Through the Real-Time PCR Method

There are four Formulas that yield healthy gums, gums with gingivitis, gums with moderate periodontitis, and gums with severe periodontitis according to the severity of the periodontal disease.

$$Formula for healthy gums = - 4.73715 + ( - 0.04148 ) \times ( P . ginivalis \% ) + ( 2.805198 ) \times ( T . forsythia \% ) + ( 2.18462 ) \times ( T . denticola \% ) + ( - 0.19053 ) \times ( P . intermedia \% ) + ( - 0.81998 ) \times ( P . endodontalis \% ) + ( 4.580745 ) \times ( F . alocis \% ) + ( 1.323154 ) \times ( F . nucleatum \% ) + ( 7.584086 ) \times ( R . dentocariosa \% ) + ( - 3.57656 ) \times ( N . subflava \% ) \qquad 1 )$$

$$Formula for gums with gingivitis = - 1.66994 + ( - 0.02906 ) \times ( P . ginivalis \% ) + ( 2.679294 ) \times ( T . forsythia \% ) + ( 1.969696 ) \times ( T . denticola \% ) + ( 0.2457071 ) \times ( P . intermedia \% ) + ( 0.025152 ) \times ( P . endodontalis \% ) + ( 5.319981 ) \times ( F . alocis \% ) + ( 0.480107 ) \times ( F . nucleatum \% ) + ( 1.29032 ) \times ( R . dentocariosa \% ) + ( 1.183753 ) \times ( N . subflava \% ) \qquad ( 2 )$$

$$Formula for gums with monderate periodontitis = - 4.57718 + ( - 0.1801 ) \times ( P . ginivalis \% ) + ( 15.36133 ) \times ( T . forsythia \% ) + ( 15.1877 ) \times ( T . denticola \% ) + ( 0.940892 ) \times ( P . intermedia \% ) + ( - 1.20232 ) \times ( P . endodontalis \% ) + ( 16.33778 ) \times ( F . alocis \% ) + ( 0.475167 ) \times ( F . nucleatum \% ) + ( - 0.52608 ) \times ( R . dentocariosa \% ) + ( 14.06681 ) \times ( N . subflava \% ) \qquad ( 3 )$$

$$Formula for gums with severe periodontitis = - 3.64997 + ( 0.225553 ) \times ( P . ginivalis \% ) + ( 0.750389 ) \times ( T . forsythia \% ) + ( - 0.09623 ) \times ( T . denticola \% ) + ( 0.265732 ) \times ( P . intermedia \% ) + ( - 0.03233 ) \times ( P . endodontalis \% ) + ( 18.2523 ) \times ( F . alocis \% ) + ( 0.526798 ) \times ( F . nucleatum \% ) + ( 0.5957 ) \times ( R . dentocariosa \% ) + ( 4.885258 ) \times ( N . subflava \% ) \qquad ( 4 )$$

After collecting the gingival crevicular fluid of the teeth to know the severity of the disease and then performing real-time PCR on the bacteria present therein using primers specific to the 9 species, the concentration % of the bacteria relative to the total bacteria for the 9 bacterial species may be calculated and then the bacterial % of the 9 species calculated may be applied to the 4 Formulas to determine the severity of gum disease corresponding to the Formula with the highest value as the severity of disease in the teeth.

When applying the % for each of the 9 bacterial species derived from real-time PCR by primers specific to the bacteria to corresponding Formula for the analyzed gingival crevicular fluid samples from the total of 51 people, including 7 teeth with healthy gums from 7 people with healthy gums, 14 teeth with gingival gums from 14 people with gingival gums, 12 teeth with moderate periodontitis from 12 people with moderate periodontitis, and 18 teeth with severe periodontitis from 18 people with severe periodontitis,

Of the 7 samples from healthy gums, 3 samples (42.86%) were analyzed as healthy gums, and 4 samples (57.14%) as gingivitis gums.

Of the 14 samples from gingivitis gums, 11 samples (78.57%) were analyzed as gingivitis gums, 1 sample (7.14%) as healthy gums, 1 sample (7.14%) as moderate periodontitis gum, and 1 sample (7.14%) as severe periodontitis gum.

Of the 12 samples from moderate periodontitis gums, 9 samples (75.00%) were analyzed as moderate periodontitis gums, and 3 samples (25.00%) as gingivitis gums.

Of the 18 samples from severe periodontitis gums, 12 samples (66.67%) were analyzed as severe periodontitis gums, 4 samples (22.22%) as gingivitis gums, and 2 samples (11.11%) as moderate periodontitis gums.

### 3. Method of Utilizing the Bacterial Count of 9 Species Obtained Through the Real-Time PCR Method

There are four Formulas that yield healthy gums, gums with gingivitis, gums with moderate periodontitis, and gums with severe periodontitis according to the severity of the disease.

$$Formula for healthy gums = - 2.09028 + ( 0.063613 ) \times ( P . ginivalis count ) + ( - 4.28627 ) \times ( T . forsythia count ) + ( - 2.29938 ) \times ( T . denticola count ) + ( 0.286028 ) \times ( P . intermedia count ) + ( - 2.59886 ) \times ( P . endodontalis count ) + ( - 1.76515 ) \times ( F . alocis count ) + ( 1.678029 ) \times ( F . nucleatum count ) + ( 63.93475 ) \times ( R . dentocariosa count ) + ( 45.44716 ) \times ( N . subflava count ) \qquad 1 )$$

$$Formula for gums with gingivitis = - 1.848 + ( 0.226929 ) \times ( P . ginivalis count ) + ( - 14.6669 ) \times ( T . forsythia count ) + ( - 10.1285 ) \times ( T . denticola count ) + ( 2.372836 ) \times ( P . intermedia count ) + ( - 9.03938 ) \times ( P . endodontalis count ) + ( - 8.53708 ) \times ( F . alocis count ) + ( 6.011738 ) \times ( F . nucleatum count ) + ( 103.446 ) \times ( R . dentocariosa count ) + ( 191.1352 ) \times ( N . subflava count ) \qquad ( 2 )$$

$$Formula for gums with monderate periodontitis = - 2.96588 + ( 0.17497 ) \times ( P . ginivalis count ) + ( - 6.25383 ) \times ( T . forsythia count ) + ( 0.027946 ) \times ( T . denticola count ) + ( 3.779165 ) \times ( P . intermedia count ) + ( - 7.31042 ) \times ( P . endodontalis count ) + ( - 8.88699 ) \times ( F . alocis count ) + ( 3.613806 ) \times ( F . nucleatum count ) + ( 34.77916 ) \times ( R . dentocariosa count ) + ( 400.9045 ) \times ( N . subflava count ) \qquad ( 3 )$$

$$Formula for gums with severe periodontitis = - 2.90144 + ( 0.682137 ) \times ( P . ginivalis count ) + ( - 45.8869 ) \times ( T . forsythia count ) + ( - 1.83537 ) \times ( T . denticola count ) + ( - 0.18553 ) \times ( P . intermedia count ) + ( 9.99311 ) \times ( P . endodontalis count ) + ( 3.639538 ) \times ( F . alocis count ) + ( 5.507213 ) \times ( F . nucleatum count ) + ( 92.53885 ) \times ( R . dentocariosa count ) + ( 203.7535 ) \times ( N . subflava count ) \qquad ( 4 )$$

After collecting the gingival crevicular fluid of the teeth to know the severity of the disease and then performing real-time PCR on the bacteria present therein using primers specific to the 9 bacterial species, the bacterial count for the 9 bacterial species may be calculated and then the bacterial count of the 9 species calculated may be applied to the 4 Formulas to determine the severity of gum disease corresponding to the Formula with the highest value as the severity of the teeth.

When applying the count for each of the 9 bacterial species derived from real-time PCR by primers specific to the bacteria to corresponding Formula for the analyzed gingival crevicular fluid samples from the total of 51 people, including 7 teeth with healthy gums from 7 people with healthy gums, 14 teeth with gingival gums from 14 people with gingival gums, 12 teeth with moderate periodontitis from 12 people with moderate periodontitis, and 18 teeth with severe periodontitis from 18 people with severe periodontitis,

7 Samples (100%) of 7 Samples from Healthy Gums were Analyzed as Gingivitis Gums.

Of the 14 samples from gingivitis gums, 13 samples (92.86%) were analyzed as gingivitis gums, and 1 sample (7.14%) as severe periodontitis gums.

Of the 12 samples from gums with moderate periodontitis, 4 samples (33.33%) were analyzed as gums with moderate periodontitis, 7 samples (58.33%) as gums with gingivitis, and 1 sample (8.33%) as severe periodontitis.

Of the 18 samples from gums with severe periodontitis, 9 samples (50.00%) were analyzed as gums with severe periodontitis, 8 samples (44.44%) as gums with gingivitis, and 1 sample (15.56%) as moderate periodontitis.

Having described specific parts of the present disclosure in detail above, it is clear to those skilled in the art that these specific descriptions are merely preferred example embodiments and do not limit the scope of the present disclosure. Thus, the substantial scope of the present disclosure will be defined by the appended claims and their equivalents.

The scope of the present disclosure is indicated by the claims described below, and all changes or modified forms derived from the meaning and scope of the claims and their equivalent concepts should be construed as being included in the scope of the present disclosure.

1. A method of providing information necessary for diagnosis of a periodontal disease, the method comprising:
performing quantitative analysis by real-time polymerase chain reaction (real-time PCR) with one or more bacteria selected from the group consisting of Porphyromonas gingivalis, Tannerella forsythia, Prevotella intermedia, Porphyromonas endodontalis, Filifactor alocis, and Treponema denticola in a sample isolated from an individual; and
comparing a bacterial % or a bacterial count obtained through the quantitative analysis by real-time PCR with a set cut-off value indicating the presence of a probing pocket greater than or equal to 5 mm that shows severity of periodontitis.

2. (canceled)

3. The method of claim 1, wherein the sample is gingival crevicular fluid.

4. The method of claim 1, wherein the periodontal disease is selected from the group consisting of severe periodontitis and recurrent periodontitis.

5. The method of claim 4, wherein the periodontal disease has a probing pocket depth greater than or equal to 5 mm.

6. The method of claim 1, wherein the quantitative analysis by real-time PCR measures an expression level of a target gene using any one or more primer sets selected from the group consisting of a primer set represented by SEQ ID NOS: 1 and 2, a primer set represented by SEQ ID NOS: 3 and 4, a primer set represented by SEQ ID NOS: 5 and 6, a primer set represented by SEQ ID NOS: 7 and 8, a primer set represented by SEQ ID NOS: 9 and 10, and a primer set represented by SEQ ID NOS: 11 and 12.

7. The method of claim 6, wherein the target gene of the primer set represented by SEQ ID NOS: 1 and 2 is rpoB in Porphyromonas gingivalis, the target gene of the primer set represented by SEQ ID NOS: 3 and 4 is rpoB in Tannerella forsythia, the target gene of the primer set represented by SEQ ID NOS: 5 and 6 is rpoB in Prevotella intermedia, the target gene of the primer set represented by SEQ ID NOS: 7 and 8 is rpoB in Porphyromonas endodontalis, the target gene of the primer set represented by SEQ ID NOS: 9 and 10 is 16s rRNA in Filifactor alocis, and the target gene of the primer set represented by SEQ ID NOS: 11 and 12 is rpoB in Treponema denticola.

8. The method of claim 1, wherein the comparing comprises determining the periodontal disease, if, based on a cut-off value of the bacterial % obtained through the quantitative analysis by real-time PCR, the cut-off value of the target gene of the primer set represented by SEQ ID NOS: 1 and 2 is greater than or equal to 0.734, the cut-off value of the target gene of the primer set represented by SEQ ID NOS: 3 and 4 is greater than or equal to 0.009, the cut-off value of the target gene of the primer set represented by SEQ ID NOS: 5 and 6 is greater than or equal to 0.041, the cut-off value of the target gene of the primer set represented by SEQ ID NOS: 7 and 8 is greater than or equal to 0.048, the cut-off value of the target gene of the primer set represented by SEQ ID NOS: 9 and 10 is greater than or equal to 0.002, or the cut-off value of the target gene of the primer set represented by SEQ ID NOS: 11 and 12 is greater than or equal to 0.002.

9. (canceled)

10. The method of claim 1, wherein the comparing comprises determining the periodontal disease, if, based on a cut-off value of the bacterial count obtained through the quantitative analysis by real-time PCR, the cut-off value of the target gene of the primer set represented by SEQ ID NOS: 1 and 2 is greater than or equal to 6489, the cut-off value of the target gene of the primer set represented by SEQ ID NOS: 3 and 4 is greater than or equal to 18, the cut-off value of the target gene of the primer set represented by SEQ ID NOS: 5 and 6 is greater than or equal to 125, the cut-off value of the target gene of the primer set represented by SEQ ID NOS: 7 and 8 is greater than or equal to 71, the cut-off value of the target gene of the primer set represented by SEQ ID NOS: 9 and 10 is greater than or equal to 3, or the cut-off value of the target gene of the primer set represented by SEQ ID NOS: 11 and 12 is greater than or equal to 23.

11. The method of claim 1, wherein the cut-off value is derived from a cut-off value of bacteria with an AUC value greater than or equal to 0.7 by obtaining an ROC curve and the AUC value for diagnosing a tooth with a probing pocket greater than or equal to 5 mm based on the count of bacteria collected from the tooth for the probing pocket of the tooth from a gingival crevicular fluid sample.

12. A method of providing information necessary for diagnosis of a periodontal disease, the method comprising:
performing 16S rRNA sequencing analysis with one or more bacteria selected from the group consisting of Porphyromonas gingivalis, Tannerella forsythia, Prevotella intermedia, Porphyromonas endodontalis, Filifactor alocis, Treponema denticola, and Rothia dentocariosa in a sample isolated from an individual; and
comparing a bacterial % obtained through the 16S rRNA sequencing analysis with a set cut-off value indicating the presence of a probing pocket greater than or equal to 5 mm that shows severity of periodontitis.

13. (canceled)

14. The method of claim 12, wherein the cut-off values are 0.170 for Porphyromonas gingivalis, 0.458 for Tannerella forsythia, 0.005 for Prevotella intermedia, 0.260 for Porphyromonas endodontalis, 0.145 for Filifactor alocis, 0.070 for Treponema denticola, and 0.240 for Rothia dentocariosa, and, in the case of Porphyromonas gingivalis, Tannerella forsythia, Prevotella intermedia, Porphyromonas endodontalis, Filifactor alocis, or Treponema denticola, a tooth is determined to have a probing pocket greater than or equal to 5 mm when it is above the cut-off value, and in the case of Rothia dentocariosa, the tooth is determined to have a probing pocket greater than or equal to 5 mm when it is below the cut-off value.

15. The method of claim 12, wherein the sample is gingival crevicular fluid.

16. The method of claim 12, wherein the periodontal disease is selected from the group consisting of severe periodontitis and recurrent periodontitis.

17. The method of claim 12, where the method further comprises:
substituting a bacterial % obtained through the 16S rRNA sequencing analysis to Equations 1-1 to 1-4 below to calculate each value and determining gum disease severity corresponding to the Equation with the highest value thereamong as severity of the disease in a tooth:
$$Formula for healthy gums = - 6.64659 + ( 0.15264 ) \times ( P . ginivalis \% ) + ( - 0.14553 ) \times ( T . forsythia \% ) + ( - 0.53978 ) \times ( T . denticola \% ) + ( - 0.00038 ) \times ( P . intermedia \% ) + ( 0.10154 ) \times ( P . endodontalis \% ) + ( - 1.16269 ) \times ( F . alocis \% ) + ( 0.35602 ) \times ( F . nucleatum \% ) + ( 0.53214 ) \times ( R . dentocariosa \% ) + ( 0.49468 ) \times ( N . subflava \% ) \qquad [ Equation 1 - 1 ]$$
$$Formula for gums with gingivitis = - 2.92299 + ( 0.13318 ) \times ( P . ginivalis \% ) + ( - 0.295 ) \times ( T . forsythia \% ) + ( - 0.35928 ) \times ( T . denticola \% ) + ( 0.04931 ) \times ( P . intermedia \% ) + ( 0.09246 ) \times ( P . endodontalis \% ) + ( - 0.62252 ) \times ( F . alocis \% ) + ( 0.28945 ) \times ( F . nucleatum \% ) + ( 0.20002 ) \times ( R . dentocariosa \% ) + ( 0.22411 ) \times ( N . subflava \% ) \qquad [ Equation 1 - 2 ]$$
$$Formula for gums with monderate periodontitis = - 3.61834 + ( 0.00999 ) \times ( P . ginivalis \% ) + ( - 0.01267 ) \times ( T . forsythia \% ) + ( 0.61089 ) \times ( T . denticola \% ) + ( 0.15731 ) \times ( P . intermedia \% ) + ( 0.17845 ) \times ( P . endodontalis \% ) + ( - 0.40155 ) \times ( F . alocis \% ) + ( 0.23367 ) \times ( F . nucleatum \% ) + ( 0.16344 ) \times ( R . dentocariosa \% ) + ( 0.30398 ) \times ( N . subflava \% ) \qquad [ Equation 1 - 3 ]$$
$$Formula for gums with severe periodontitis = - 5.6885 + ( 0.33528 ) \times ( P . ginivalis \% ) + ( - 0.08898 ) \times ( T . forsythia \% ) + ( - 0.80137 ) \times ( T . denticola \% ) + ( 0.05997 ) \times ( P . intermedia \% ) + ( 0.21088 ) \times ( P . endodontalis \% ) + ( - 1.11862 ) \times ( F . alocis \% ) + ( 0.40108 ) \times ( F . nucleatum \% ) + ( 0.26583 ) \times ( R . dentocariosa \% ) + ( 0.33004 ) \times ( N . subflava \% ) \qquad [ Equation 1 - 4 ]$$

18. The method of claim 1, wherein the method further comprises:
substituting a bacterial % obtained through the quantitative analysis by real-time PCR to Equations 2-1 to 2-4 below to calculate each value and determining gum disease severity corresponding to the Equation with the highest value thereamong as severity of the disease in a tooth:
$$Formula for healthy gums = - 4.73715 + ( - 0.04148 ) \times ( P . ginivalis \% ) + ( 2.805198 ) \times ( T . forsythia \% ) + ( 2.18462 ) \times ( T . denticola \% ) + ( - 0.19053 ) \times ( P . intermedia \% ) + ( - 0.81998 ) \times ( P . endodontalis \% ) + ( 4.580745 ) \times ( F . alocis \% ) + ( 1.323154 ) \times ( F . nucleatum \% ) + ( 7.584086 ) \times ( R . dentocariosa \% ) + ( - 3.57656 ) \times ( N . subflava \% ) \qquad [ Equation 2 - 1 ]$$
$$Formula for gums with gingivitis = - 1.66994 + ( - 0.02906 ) \times ( P . ginivalis \% ) + ( 2.679294 ) \times ( T . forsythia \% ) + ( 1.969696 ) \times ( T . denticola \% ) + ( 0.2457071 ) \times ( P . intermedia \% ) + ( 0.025152 ) \times ( P . endodontalis \% ) + ( 5.319981 ) \times ( F . alocis \% ) + ( 0.480107 ) \times ( F . nucleatum \% ) + ( 1.29032 ) \times ( R . dentocariosa \% ) + ( 1.183753 ) \times ( N . subflava \% ) \qquad [ Equation 2 - 2 ]$$
$$Formula for gums with monderate periodontitis = - 4.57718 + ( - 0.1801 ) \times ( P . ginivalis \% ) + ( 15.36133 ) \times ( T . forsythia \% ) + ( 15.1877 ) \times ( T . denticola \% ) + ( 0.940892 ) \times ( P . intermedia \% ) + ( - 1.20232 ) \times ( P . endodontalis \% ) + ( 16.33778 ) \times ( F . alocis \% ) + ( 0.475167 ) \times ( F . nucleatum \% ) + ( - 0.52608 ) \times ( R . dentocariosa \% ) + ( 14.06681 ) \times ( N . subflava \% ) \qquad [ Equation 2 - 3 ]$$
$$Formula for gums with severe periodontitis = - 3.64997 + ( 0.225553 ) \times ( P . ginivalis \% ) + ( 0.750389 ) \times ( T . forsythia \% ) + ( - 0.09623 ) \times ( T . denticola \% ) + ( 0.265732 ) \times ( P . intermedia \% ) + ( - 0.03233 ) \times ( P . endodontalis \% ) + ( 18.2523 ) \times ( F . alocis \% ) + ( 0.526798 ) \times ( F . nucleatum \% ) + ( 0.5957 ) \times ( R . dentocariosa \% ) + ( 4.885258 ) \times ( N . subflava \% ) \qquad [ Equation 2 - 4 ]$$

19. The method of claim 1, wherein the method further comprises:
substituting a bacterial count obtained through the quantitative analysis by real-time PCR to Equations 3-1 to 3-4 below to calculate each value and determining gum disease severity corresponding to the Equation with the highest value thereamong as severity of the disease in a tooth:
$$Formula for healthy gums = - 2.09028 + ( 0.063613 ) \times ( P . ginivalis count ) + ( - 4.28627 ) \times ( T . forsythia count ) + ( - 2.29938 ) \times ( T . denticola count ) + ( 0.286028 ) \times ( P . intermedia count ) + ( - 2.59886 ) \times ( P . endodontalis count ) + ( - 1.76515 ) \times ( F . alocis count ) + ( 1.678029 ) \times ( F . nucleatum count ) + ( 63.93475 ) \times ( R . dentocariosa count ) + ( 45.44716 ) \times ( N . subflava count ) \qquad [ Equation 3 - 1 ]$$
$$Formula for gums with gingivitis = - 1.848 + ( 0.226929 ) \times ( P . ginivalis count ) + ( - 14.6669 ) \times ( T . forsythia count ) + ( - 10.1285 ) \times ( T . denticola count ) + ( 2.372836 ) \times ( P . intermedia count ) + ( - 9.03938 ) \times ( P . endodontalis count ) + ( - 8.53708 ) \times ( F . alocis count ) + ( 6.011738 ) \times ( F . nucleatum count ) + ( 103.446 ) \times ( R . dentocariosa count ) + ( 191.1352 ) \times ( N . subflava count ) \qquad [ Equation 3 - 2 ]$$
$$Formula for gums with monderate periodontitis = - 2.96588 + ( 0.17497 ) \times ( P . ginivalis count ) + ( - 6.25383 ) \times ( T . forsythia count ) + ( 0.027946 ) \times ( T . denticola count ) + ( 3.779165 ) \times ( P . intermedia count ) + ( - 7.31042 ) \times ( P . endodontalis count ) + ( - 8.88699 ) \times ( F . alocis count ) + ( 3.613806 ) \times ( F . nucleatum count ) + ( 34.77916 ) \times ( R . dentocariosa count ) + ( 400.9045 ) \times ( N . subflava count ) \qquad [ Equation 3 - 3 ]$$
$$Formula for gums with severe periodontitis = - 2.90144 + ( 0.682137 ) \times ( P . ginivalis count ) + ( - 45.8869 ) \times ( T . forsythia count ) + ( - 1.83537 ) \times ( T . denticola count ) + ( - 0.18553 ) \times ( P . intermedia count ) + ( 9.99311 ) \times ( P . endodontalis count ) + ( 3.639538 ) \times ( F . alocis count ) + ( 5.507213 ) \times ( F . nucleatum count ) + ( 92.53885 ) \times ( R . dentocariosa count ) + ( 203.7535 ) \times ( N . subflava count ) \qquad [ Equation 3 - 4 ]$$

20-21. (canceled)
